(19) United States
(12) Patent Application Publication
(10) Pub. No.: US 2011/0060243 A1
(43) Pub. Date: Mar. 10, 2011

(54) Systems and methods for intra-operative regional neural stimulation

(75) Inventors: Michael R. Hausman, New York, NY (US); Geoffrey B. Thrope, Shaker Heights, OH (US); Robert B. Strother, Willoughby Hills, OH (US); Joseph J. Mrva, Euclid, OH (US)

(73) Assignee: Checkpoint Surgical, LLC

(21) Appl. No.: 12/806,698

(22) Filed: Aug. 19, 2010

Related U.S. Application Data
(63) Continuation-in-part of application No. 11/651,165, filed on Jan. 9, 2007.
Application No. 11/651,165 is a continuation-in-part of application No. 11/099,848, filed on Apr. 6, 2005.
(60) Provisional application No. 60/657,277, filed on Mar. 1, 2005.
(60) Provisional application No. 61/338,312, filed on Feb. 16, 2010.

(51) Int. Cl. A61B 5/05

(52) U.S. Cl. 600/554

(57) ABSTRACT

Systems and methods according to the present invention provide a means for determining, as an example, the health or location of nerves. Generally, embodiments according to the present invention may be used to verify the absence of nerve tissue along a prospective incision location to avoid damage caused by such incision. Alternatively or additionally, embodiments according to the present invention may be used to locate and/or hone in on a more precise location of one or more nerves. Electrical stimulation may be applied to tissue in an iterative fashion in one or more desired patterns and at one or more stimulation intensities to determine one or more regional neural responses and one or more focused neural responses.

### RELATED APPLICATIONS

This application is a continuation-in-part of co-pending U.S. patent application Ser. No. 11/651,165, filed Jan. 9, 2007, and entitled “Systems and Methods for Intra-Operative Stimulation,” which is a continuation-in-part of U.S. patent application Ser. No. 11/099,848, filed Apr. 6, 2005, and entitled “Systems and Methods for Intra-Operative Stimulation,” which claims the benefit of U.S. Provisional Patent Application Ser. No. 60/657,277, filed Mar. 1, 2005, and entitled “Systems and Methods for Intra-Operative Stimulation,” each of which is incorporated herein by reference in its entirety.

This application also claims the benefit of U.S. Patent Application Ser. No. 61/338,312, filed Feb. 16, 2010, and entitled “Systems and Methods for Intra-Operative Stimulation,” which is incorporated herein by reference in its entirety.

### FIELD OF THE INVENTION

The invention relates generally to tissue identification and integrity testing, and more particularly to systems and methods for safeguarding against nerve and muscle injury during surgical procedures, location and stimulation of nerves and muscles, identification and assessment of nerve and muscle integrity following traumatic injuries, and verification of range of motion and attributes of muscle contraction during reconstructive surgery.

### BACKGROUND OF THE INVENTION

Even with today's sophisticated medical devices, surgical procedures are not risk-free. Each patient's anatomy differs, requiring the surgeon to be ever vigilant to these differences so that the intended result is accomplished. The positioning of nerves and other tissues within a human or animal's body is one example of how internal anatomy differs from patient to patient. While these differences may be slight, if the surgeon fails to properly identify one or several nerves, the nerves may be bruised, stretched, or even severed during an operation. The negative effects of nerve damage can range from lack of feeling on that part of the body to loss of muscle control.

Traumatic injuries often require surgical repair. Determining the extent of muscle and nerve injury is not always possible using visual inspection. Use of an intra-operative stimulator enables accurate evaluation of the neuromuscular system in that area. This evaluation provides valuable knowledge to guide repair/reconstructive surgery following traumatic injury, and when performing a wide range of surgeries.

### SUMMARY OF THE INVENTION

The invention provides devices, systems, and methods for intra-operative stimulation. The intra-operative stimulation enables accurate evaluation of the neuromuscular system to guide repair or reconstructive surgery.

An embodiment of a method according to the present invention includes a method of avoiding nerve tissue in a surgical procedure. The targeted tissue In a first application step, a first electrical stimulation is applied with an electrode to a first tissue region along the first potential incision length. The first tissue region may be cutaneous or subcutaneous. During the first application step, a neural response may be observed. In the event that a neural response is observed, a second incision location and a second potential incision length may be identified, so as to avoid damage to the nerve which was stimulated during the first application step. In a second application step, a second electrical stimulation may be applied with the electrode to a second tissue region along the second potential incision length. During the second application, a neural response may or may not be observed. If no neural response is observed, a surgical procedure may be performed on or through the second tissue region. The surgical procedure may include the step of incising a portion of the second tissue region along a portion of the second potential incision length.

According to one aspect of a method according to the present invention, if no neural response is observed in response to a stimulation, a parameter of the stimulation may be adjusted and, in a third application step, a third electrical stimulation may be applied with an electrode to the first tissue region along the first potential incision length. The adjustment to the stimulation parameter may be a decrease in stimulation intensity by reducing one or both of stimulation pulse duration and stimulation amplitude. Alternatively, the adjustment to the stimulation parameter may be an increase in stimulation intensity by increasing one or both of stimulation pulse duration and stimulation amplitude.

Another embodiment of a method according to the present invention includes a method of locating nerve tissue to perform a surgical procedure, perhaps in the area of or on the nerve tissue. The method includes identifying a first potential incision location and a first potential incision length. In a first application step, a first electrical stimulation is applied with an electrode to a tissue region along the first potential incision length. The tissue region may include tissue that is cutaneous or subcutaneous. During the first application step, a neural response may or may not be observed. If no desired neural response is observed, a parameter of the electrical stimulation may be altered, and, in a second application step, a second stimulation may be applied with the electrode to the tissue region. During the second application step, a neural response may or may not be observed. If a neural response is observed, after the first and second application steps, an incision may be made along at least a portion of the first potential incision length, and a surgical procedure may be performed through or accomplished by the incision. The method may further include, in a third application step, applying the first stimulation to the tissue region, and observing the desired neural response during the third application step.

According to an aspect of a method according to this embodiment, the first application step may be performed before the altering step and the second application step may be performed after the altering step. The altering step may include the step of increasing or decreasing electrical stimulation intensity, which may be accomplished by increasing or decreasing, respectively, at least one of electrical stimulation amplitude and/or pulse duration.

According to another aspect of a method according to the present invention, the application steps may include the step of translating the electrode along at least a portion of an incision length while the electrode is in contact with the tissue. Thus, the electrical stimulation may be applied while the electrode is moving in contact with the tissue.

According to yet another aspect of a method according to the present invention, the surgical procedure may include the step of removing scar tissue, which may be removed through or caused by the incision. In one embodiment two devices are used for stimulation and surgery, respectively. For instance embodiments of systems disclosed herein may be used for electrical stimulation, and a scalpel may be used for performing the surgical procedure.

Another embodiment of a method according to the present invention is a method for honing in on nerve fibers disposed below or innervating animal tissue, which may be cutaneous or subcutaneous tissue. The method includes the step of applying a first electrical stimulation to animal tissue, at a first stimulation intensity, within an identified stimulation region, the first electrical stimulation being applied with an electrode in contact with the tissue. A plurality of first active stimulation locations may be identified within the stimulation region. The active stimulation locations are locations of the electrode in contact with tissue at which one or more neural responses are generated in response to the first electrical stimulation. The plurality of first active stimulation locations is disposed about a perimeter of a focused stimulation region. At a reduced stimulation intensity, a second electrical stimulation may be applied within the focused stimulation region. In a second identifying step, at least one second active stimulation location may be identified within the focused stimulation region. The reduced stimulation intensity may be generated by a step of altering an electrical stimulation parameter of the electrical stimulation. The step of altering may include reducing at least one of electrical stimulation pulse duration and electrical stimulation pulse amplitude.

According to an aspect of such embodiment, the first identifying step may include the step of translating the electrode across the tissue while applying the first electrical stimulation. The electrode may be translated in a desired pattern, such as a star pattern, a zig-zag pattern, or a spiral pattern.

According to another aspect of an embodiment of honing in on nerve fibers, the second identifying step may include the step of translating the electrode across the tissue while applying the second electrical stimulation. The electrode may be translated in a desired pattern, such as a star pattern, a zig-zag pattern, or a spiral pattern.

Yet another embodiment of a method according to the present invention includes a method of seeking a neural response to electrical stimulation. The method includes the step of identifying a prospecting location on animal tissue, which may be cutaneous or subcutaneous. This location may be selected based on prior experience with other patients or prior experience with a specific patient. Alternatively, the location may be randomly selected. An electrical stimulation maybe applied to the tissue at the prospecting location with an electrode to determine whether a neural response is generated. A neural response may or may not be observed.

If no neural response is observed, the electrode may be translated while in contact with the tissue radially outward from the prospecting location in a first direction for a first honing distance. The method may further include the step of translating the electrode in a second direction back to the prospecting location. The electrode may then be translated radially outward from the prospecting location in a third direction for a second honing distance, and a neural response to the electrical stimulation may be observed. The third direction may be substantially the same as the second direction, and the second honing distance may be substantially the same as the first honing distance.

According to an aspect of such embodiment, the step of translating in the first direction may be performed while applying the electrical stimulation to the tissue. Alternatively or additionally, one or more of the translating steps may be performed while the electrode is in contact with the tissue. The second direction may be opposite the first direction.

According to another aspect of an embodiment of seeking a neural response, the method may include the step of identifying an active stimulation location on the tissue.

According to yet another aspect of an embodiment of seeking a neural response, one or more of the following steps may be repeated: identifying a prospecting location; applying electrical stimulation to the tissue at the prospecting location with an electrode to determine whether a neural response is generated; and identifying an active stimulation location on the tissue.

Features and advantages of the inventions are set forth in the following Description and Drawings, as well as the appended description of technical features.

### BRIEF DESCRIPTION OF THE DRAWINGS

FIG. 1 is a diagrammatic view of a system usable in association with a family of different monitoring and treatment devices for use in different medical procedures.

FIG. 2 is a perspective view showing an exemplary embodiment of the system shown in FIG. 1, the stimulation control device being removably coupled to a stimulation probe, and showing the stimulation signal path through the system.

FIG. 3A is a side view with a portion broken away and in section showing the stimulation probe having the stimulation control device embedded within the stimulation probe.

FIG. 3B is a side view with a portion broken away and in section showing the stimulation probe having the stimulation control device embedded within the stimulation probe, and showing an optional needle-like return electrode.

FIG. 3C is a side view with a portion broken away and in section showing an additional embodiment of the stimulation probe having a housing that includes a gripping base and a flexible nose cone, and an illuminating ring indicator.

FIG. 4A is a side view of the stimulation probe of FIG. 3c, showing the users hand in a position on the stimulation probe to move the flexible nose cone.

FIG. 4B is a side view of the stimulation probe of FIG. 4A, showing the users hand flexing the flexible nose cone.

FIG. 5 is a side view with a portion broken away and in section showing elements of the flexible nose cone, the ring indicator, and the gripping base.

FIG. 6 is a graphical view of a desirable biphasic stimulus pulse output of the stimulation device.

FIG. 7 is a view showing how the geometry of the stimulation control device shown in FIG. 2 aids in its positioning during a surgical procedure.

FIG. 8 is a block diagram of a circuit that the stimulation control device shown throughout the Figs. can incorporate.

FIGS. 9A and 9B are perspective views showing the stimulation control device in use with a cutting device.

FIGS. 10A and 10B are perspective views showing the stimulation control device in use with a drilling or screwing device.

FIGS. 11A and 11B are perspective views showing the stimulation control device in use with a pilot auger device.

FIGS. 12A and 12B are perspective views showing the stimulation control device in use with a fixation device.

FIG. 13 is a plane view of a kit used in conjunction with the stimulation probe shown in FIG. 3C, and including the stimulation probe and instructions for use.

FIG. 14 is a perspective view of the stimulation probe shown in FIG. 3C.

FIG. 15 is an exploded view of the stimulation probe shown in FIG. 14.

FIG. 16 is a flow chart of a first embodiment of a method according to the present invention.

FIG. 17 is a flow chart of a second embodiment of a method according to the present invention.

FIGS. 18A-18F depict a first series of steps according to the embodiment of FIG. 17.

FIG. 19 depicts an alternate stimulation, relocation and identification pattern to be used in the embodiment of FIG. 17.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF PREFERRED EMBODIMENTS

This Specification discloses various systems and methods for safeguarding against nerve, muscle, and tendon injury during surgical procedures or confirming the identity and/or location of nerves, muscles, and tendons and evaluating their function or the function of muscles enervated by those nerves. The systems and methods are particularly well suited for assisting surgeons in identification of nerves and muscles in order to assure nerve and muscle integrity during medical procedures using medical devices such as stimulation monitors, cutting, drilling, and screwing devices, pilot augers, and fixation devices. For this reason, the systems and methods will be described in the context of these medical devices.

The systems and methods desirably allow the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Still, it should be appreciated that the disclosed systems and methods are applicable for use in a wide variety of medical procedures with a wide variety of medical devices. By way of non-limiting example, the various aspects of the invention have application in procedures requiring grasping medical devices and internal viewing devices as well.

### I. Overview of the System

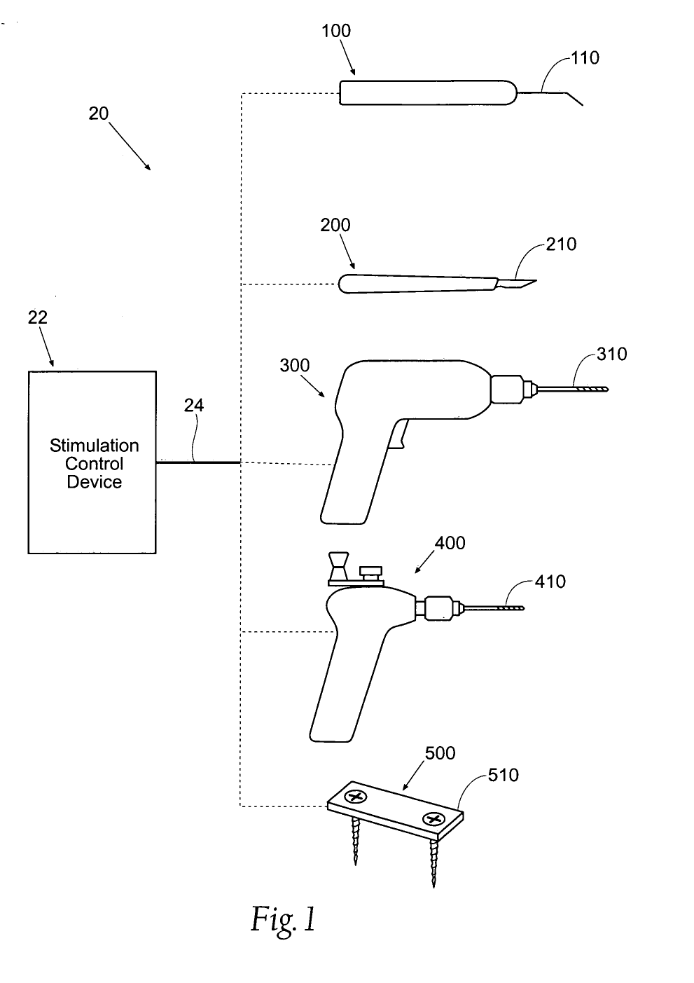

FIG. 1 shows an illustrative system 20 for locating and identifying tissue and safeguarding against tissue and/or bone injury during surgical procedures. In the illustrated embodiment, the system 20 is configured for locating, monitoring, and stimulating tissue and other structures throughout the body. The system 20 includes a stimulation control device 22 operating individually or in conjunction with one or more of a family of stimulating medical devices including, for example, a stimulation monitor or probe 100, a cutting device 200, a drilling or screwing device 300, a pilot auger 400, and a fixation device 500.

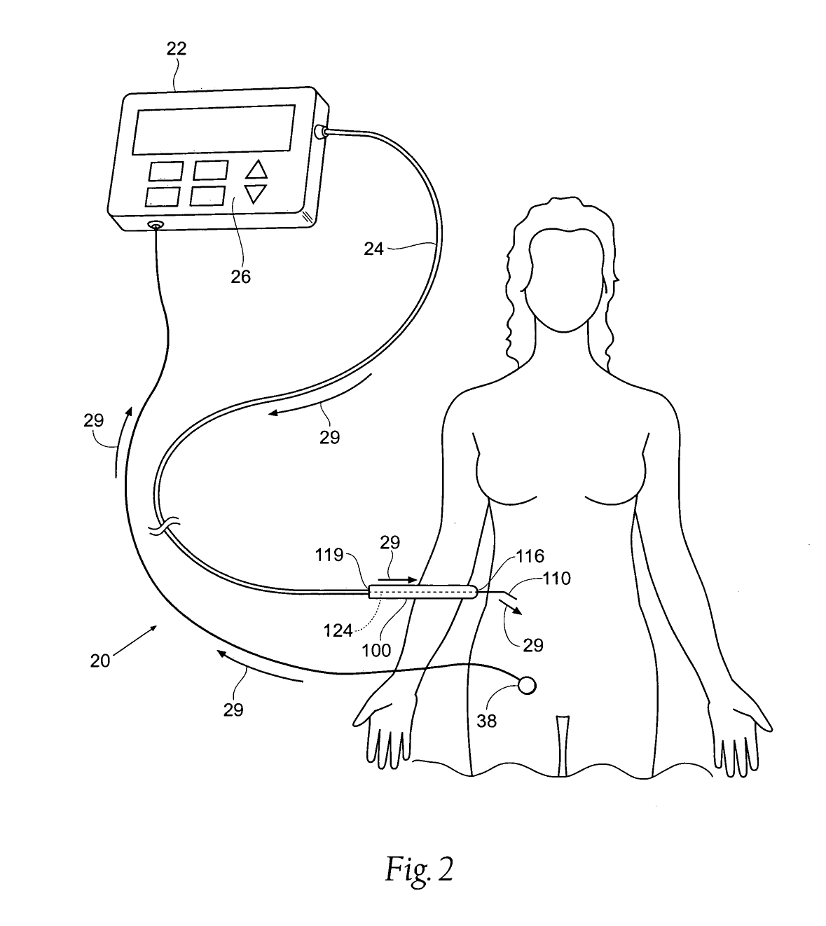

In an exemplary embodiment, and as can be seen in FIG. 2, the stimulation control device 22 functions in the system 20 to generate an electrical stimulation signal 29. The stimulation signal 29 flows from the stimulation control device 22 through a lead 24 to a medical device (e.g., stimulation probe 100). The stimulation signal 29 then flows through a predefined insulated path 124 within the stimulation probe 100 and to an operative element, such as an electrically conductive surface, i.e., a coupled electrode 110. The electrode 110 is to be positioned on or near a region of a patient to be stimulated. In monopolar operation, a return electrode (or indifferent electrode) 38 provides an electrical path from the body back to the control device 22. The stimulation control device 22 may operate in a monopolar or bipolar configuration, as will be described in greater detail later.

The stimulation signal 29 is adapted to provide an indication or status of the device. The indication may include a physical motor response (e.g., twitching), and/or one or more visual or audio signals from the stimulation control device 22, which indicate to the surgeon the status of the device, and/or close proximity of the electrode 110 to a nerve, or a muscle, or a nerve and a muscle. The stimulation control device may also indicate to the surgeon that the stimulation control device is operating properly and delivering a stimulus current.

### II. Medical Devices

The configuration of the stimulating medical devices that form a part of the system can vary in form and function. Various representative embodiments of illustrative medical devices will be described.

A. Stimulation Probe

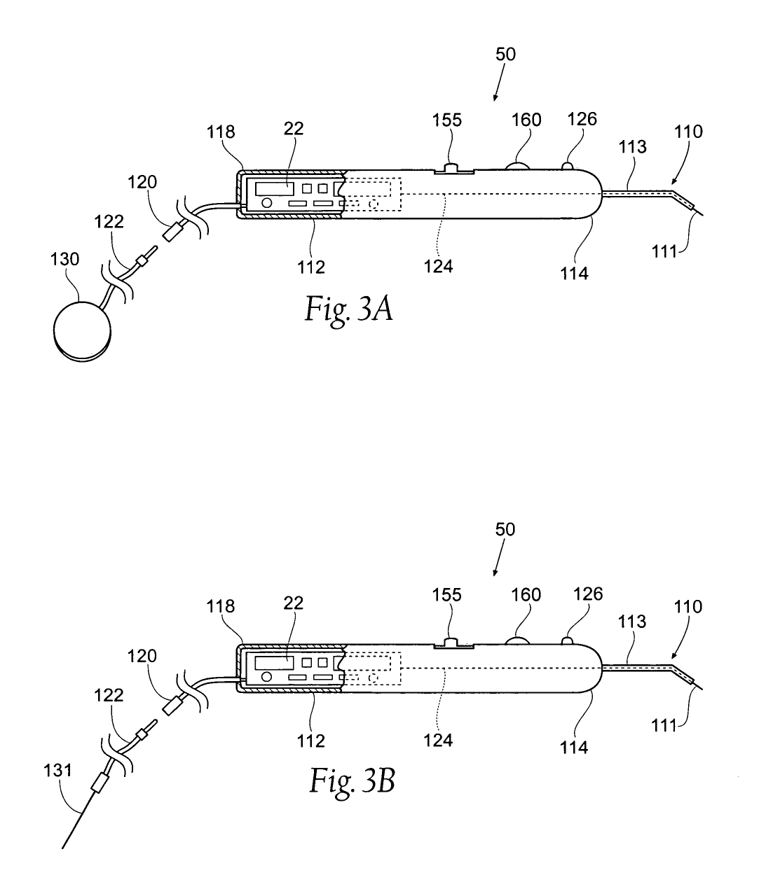

FIGS. 3A to 3C show various embodiments of a hand held stimulation monitor or probe 50 for identification and testing of nerves and/or muscles during surgical procedures. As shown, the stimulation probe 50 may accommodate within a generally tubularly housing 112 the electrical circuitry of a stimulation control device 22. The stimulation probe 50 is desirably an ergonomic, sterile, single use instrument intended for use during surgical procedures to identify nerves and muscles, muscle attachments, or to contract muscles to assess the quality of surgical interventions or the need for surgical interventions, or to evaluate the function of nerves already identified through visual means. The stimulation probe 50 may be sterilized using ethylene oxide, for example.

The stimulation probe 50 is preferably sized small enough to be held and used by one hand during surgical procedures, and is ergonomically designed for use in either the left or right hand. In a representative embodiment, the stimulation probe 50 may have a width of about 20 millimeters to about 30 millimeters, and desirably about 25 millimeters. The length of the stimulation probe 50 (not including the operative element 110) may be about 18 centimeters to about 22 centimeters, and desirably about 20 centimeters. The operative element 110 may also include an angle or bend to facilitate access to deep as well as superficial structures without the need for a large incision. The operative element 110 will be described in greater detail later. A visual or audio indicator 126 incorporated with the housing 112 provides reliable feedback to the surgeon as to the request and delivery of stimulus current.

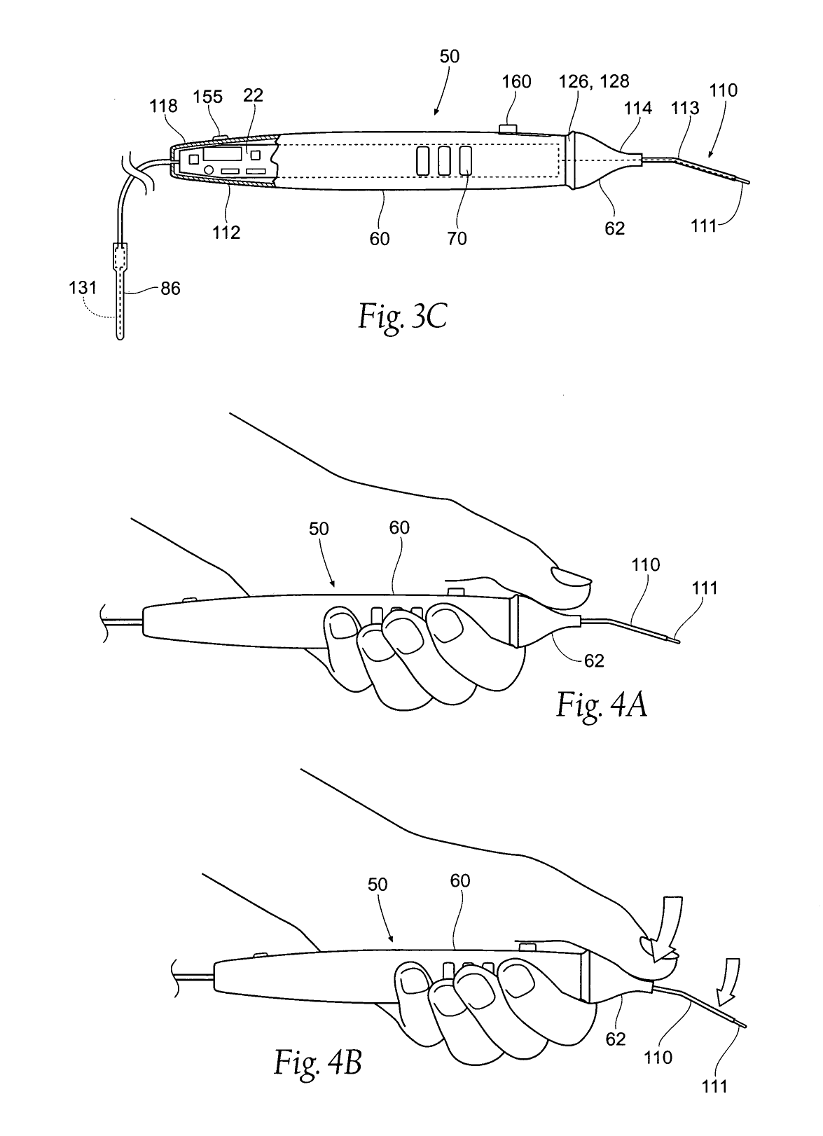

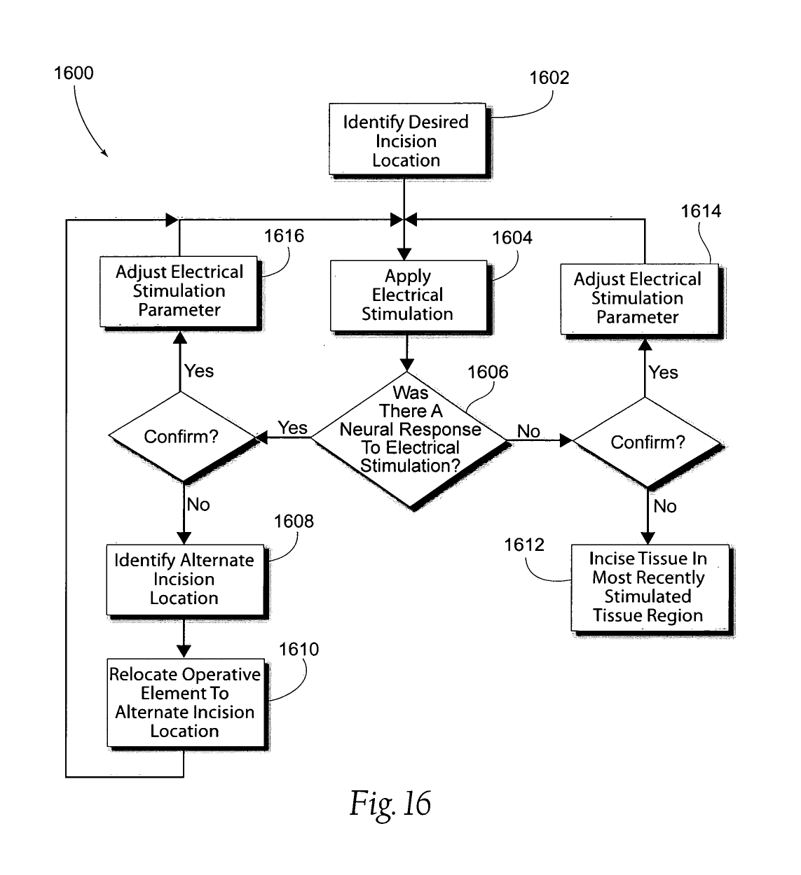

In one embodiment shown in FIGS. 3C and 14, the stimulation probe 50 includes a housing 112 that comprises a gripping base portion 60 and an operative element adjustment portion 62. The operative element 110 extends from the proximal end of the adjustment portion 62. In order to aid the surgeon in the placement of the operative element 110 at the targeted tissue region, the adjustment portion, as will be described as a nose cone 62, may be flexible. This flexibility allows the surgeon to use either a finger or a thumb positioned on the nose cone 62 to make fine adjustments to the position of stimulating tip 111 of the operative element 110 at the targeted tissue region (see FIGS. 4A and 4B). The surgeon is able to grasp the gripping base 60 with the fingers and palm of the hand, and position the thumb on the nose cone 62, and with pressure applied with the thumb, cause the stimulating tip 111 to move while maintaining a steady position of the gripping base portion 62. This flexible nose cone 62 feature allows precise control of the position of the stimulating tip 111 with only the movement of the surgeon's thumb (or finger, depending on how the stimulating probe is held).

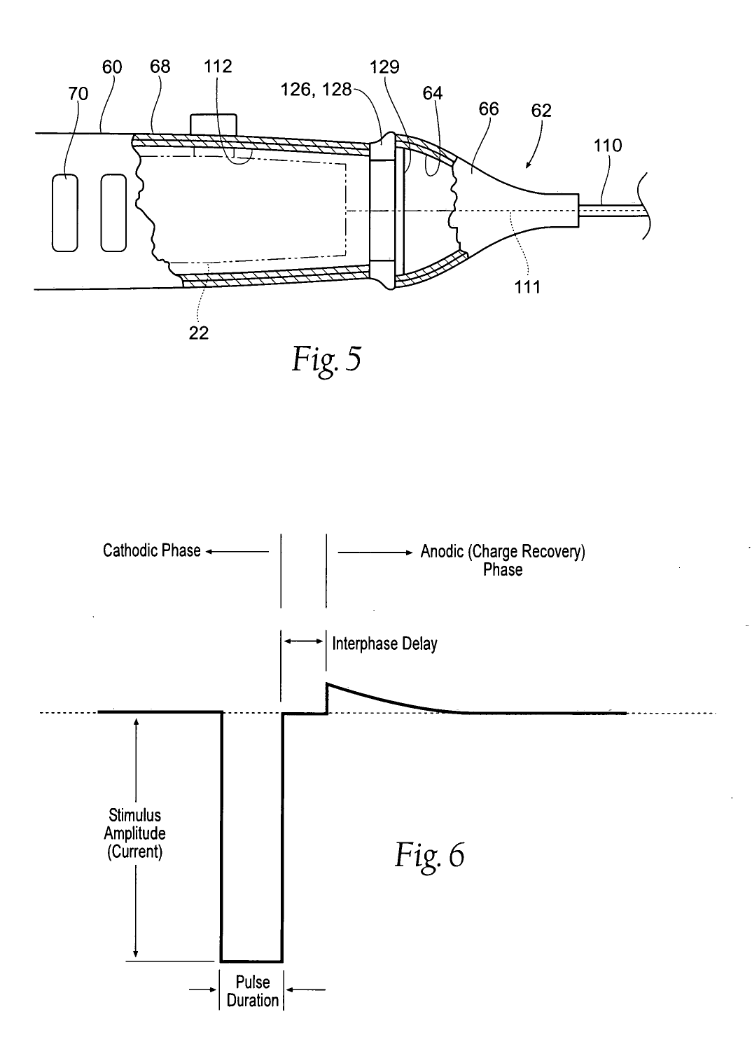

The flexible nose cone 62 may comprise a single element or it may comprise at least an inner portion 64 and an outer portion 66, as shown in FIG. 5. In order to facilitate some flexibility of the proximal portion 114 of the stimulation probe 50, the inner portion 64 of the nose cone 62 may be made of a thermoplastic material having some flexibility. One example may be LUSTRAN® ABS 348, or similar material. The outer portion 66 may comprise a softer over molded portion and may be made of a thermoplastic elastomer material having some flexibility. One example may be VERSAFLEX™ OM 3060-1 from GLS Corp. The nose cone 62 is desirably generally tapered. For example, the nose cone 62 may be rounded, as shown in FIGS. 3A and 3B, or the nose cone may be more conical in shape, as shown in FIG. 3C.

The nose cone 62 may also include one or more features, such as ribs or dimples 72, as shown in FIG. 14, to improve the gripping, control, and stability of the stimulation probe 50 within the surgeon's hand.

The gripping base portion 60 of the housing 112 may also include an overmolded portion 68. The overmolded portion 68 may comprise the full length of the gripping base portion 60, or only a portion of the gripping base 60. The soft overmolded portion 68 may include one or more features, such as dimples or ribs 70, as shown, to improve the gripping, control, and stability of the stimulation probe 50 within the surgeon's hand. The overmolded portion 68 may comprise the same or similar material as the thermoplastic elastomer material used for the outer portion 66 of the flexible nose cone 62.

In one embodiment, the stimulation probe 50 includes a housing 112 that carries an insulated lead 124. The insulated lead 124 connects the operative element 110 positioned at the housing's proximal end 114 to the circuitry 22 within the housing 112 (see FIG. 3A). It is to be appreciated that the insulated lead is not necessary and the operative element 110 may be coupled to the circuitry 22 (see FIG. 3C). The lead 124 within the housing 112 is insulated from the housing 112 using common insulating means (e.g., wire insulation, washers, gaskets, spacers, bushings, and the like). The conductive tip 111 of the operative element 110 is positioned in electrical conductive contact with at least one muscle, or at least one nerve, or at least one muscle and nerve.

As shown, the stimulation probe 50 is mono-polar and is equipped with a single operative element (i.e., electrode) 110 at the housing proximal end 114. A return electrode 130, 131 may be coupled to the stimulation probe 50 and may be any of a variety of electrode types (e.g., paddle, needle, wire, or surface), depending on the surgical procedure being performed. As shown, the various return electrodes 130, 131 are coupled to the housing distal end 118. In an alternative embodiment, the stimulation device 50 itself may be bipolar by including a return electrode in the operative element 110, which precludes the use of a return electrode coupled to the stimulation probe 50.

As shown and described, the stimulation probe 50 may accommodate within the housing 112 the electrical circuitry of a stimulation control device 22. In this arrangement, the stimulation probe 50 may have one or more user operable controls. Two are shown—155 and 160. Power switch 155 serves a dual purpose of turning the stimulation probe 500N and OFF (or standby), and also can be stepped to control the stimulation signal amplitude selection within a predefined range (e.g., 0.5, 2.0, and 20 mA). In this configuration, the switch may be a four position switch. Before the first use of the stimulation probe 50, the power switch 155 is in the OFF position and keeps the stimulation probe off. After the stimulation probe 50 has been turned ON—by moving the switch 155 to an amplitude selection—the OFF position now corresponds to a standby condition, where no stimulation would be delivered. In one embodiment, once the stimulation probe 50 has been turned on, it cannot be turned off, it can only be returned to the standby condition and will remain operational for a predetermined time, e.g., at least about seven hours. This feature is intended to allow the stimulation probe 50 to only be a single use device, so it can not be turned OFF and then used again at a later date.

The pulse control device 160 allows for adjustment of the stimulation signal pulse width from a predefined range (e.g., about zero to about 200 microseconds). In one embodiment, the pulse control 160 may be a potentiometer to allow a slide control to increase or decrease the stimulation signal pulse width within the predefined range.

The stimulation pulse may have a non-adjustable frequency in the range of about 10 Hz to about 20 Hz, and desirably about 16 Hz.

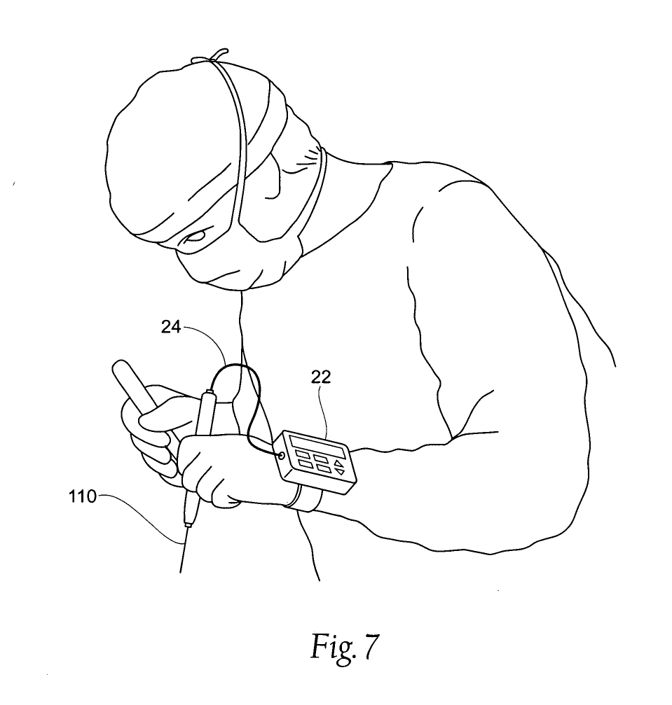

As a representative example, the stimulation pulse desirably has a biphasic waveform with controlled current during the cathodic (leading) phase, and net DC current less than 10 microamps, switch adjustable from about 0.5 milliamps to about 20 milliamps, and pulse durations adjustable from about zero microseconds up to about 200 microseconds. A typical, biphasic stimulus pulse is shown in FIG. 6.

The operative element 110 exits the housing 112 at the proximal end 114 to deliver stimulus current to the excitable tissue. The operative element 110 comprises a length and a diameter of a conductive material, and is desirably fully insulated with the exception of the most proximal end, e.g. about 1.0 millimeters to about 10 millimeters, and desirably about 4 millimeters to about 6 millimeters, which is non-insulated and serves as the stimulating tip or surface (or also referred to as active electrode) 111 to allow the surgeon to deliver the stimulus current only to the intended tissue. The small area of the stimulating surface 111 (the active electrode) of the operative element 110 ensures a high current density that will stimulate nearby excitable tissue. The insulation material 113 may comprise a medical grade heat shrink.

The conductive material of the operative element 110 comprises a diameter having a range between about 0.5 millimeters to about 1.5 millimeters, and may be desirably about 1.0 millimeters. The length of the operative element 110 may be about 50 millimeters to about 60 millimeters, although it is to be appreciated that the length may vary depending on the particular application. As shown, the operative element 110 may include one or more bends to facilitate accurate placement of the stimulating surface 111. In one embodiment, the conductive material of operative element 110 is made of a stainless steel 304 solid wire, although other known conductive materials may be used.

As previously described, in monopolar operation, a return electrode (or indifferent electrode) 130 or 131, for example, provides an electrical path from the body back to the control device 22 within the housing 112. The return electrode 130 (see FIG. 3A) may be placed on the surface of intact skin (e.g., surface electrodes as used for ECG monitoring during surgical procedures) or it might be needle-like 131 (see FIGS. 3B and 3C), and be placed in the surgical field or penetrate through intact skin. The housing's distal end 118 can incorporate a connector or jack 120 which provides options for return current pathways, such as through a surface electrode 130 or a needle electrode 131, having an associated plug 122. It is to be appreciated that a return electrode and associated lead may be an integral part of the stimulation probe 50, i.e., no plug or connector, as shown in FIG. 3C.

Additionally, the device 50 may desirably incorporate a visual or audio indicator 126 for the surgeon. This visual or audio indicator 126 allows the surgeon to confirm that the stimulator 50 is delivering stimulus current to the tissue it is contacting. Through the use of different tones, colors, different flash rates, etc., the indicator 126 (which can take the form, e.g., of a light emitting diode (LED)) allows the surgeon to confirm that the stimulating tip 111 is in place, the instrument is turned ON, and that stimulus current is flowing. Thus the surgeon has a much greater confidence that the failure to elicit a muscle contraction is because of lack of viable nervous tissue near the tip 111 of the stimulator 50 rather than the failure of the return electrode connection or some other instrumentation problem.

As a representative example, in use the indicator 126 may be configured to illuminate continuously in one color when the stimulation probe 50 is turned on but not in contact with tissue. After contact with tissue is made, the indicator 126 may flash (i.e., blink) to indicate that stimulation is being delivered. If the stimulation has been requested, i.e., the stimulation probe has been turned on, but there is no stimulation being delivered because of a lack of continuity between the operative element 110 and the return electrode 130, or an inadequate connection of the operative element 110 or the return electrode 130 to the patient tissue, the indicator 126 may illuminate in a different color, and may illuminate continuously or may flash.

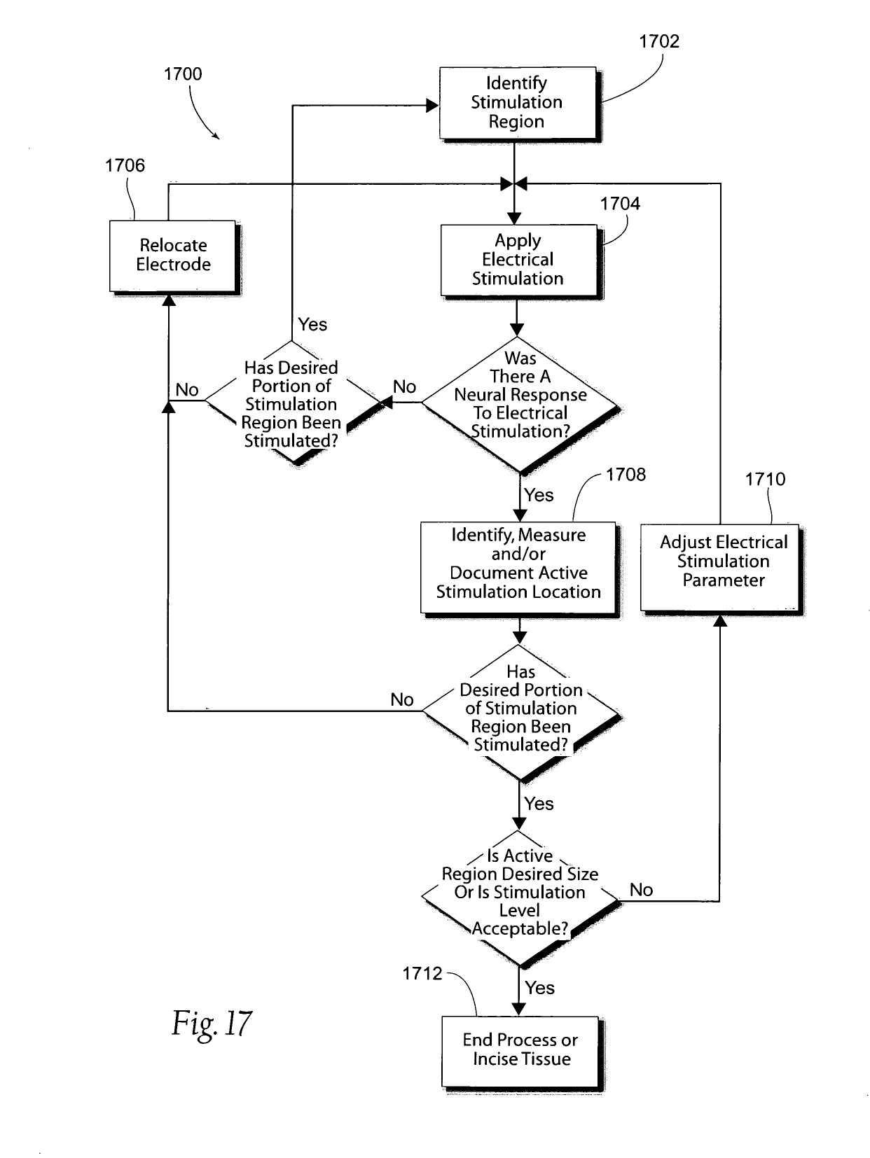

In one embodiment, as can be best seen in FIGS. 3C and 5, the indicator 126 comprises a ring indicator 128 that provides a visual indication around at least a portion, and desirably all of the circumference of the stimulation probe 50 generally near the flexible nose cone 62. The visual ring indicator 128 may be an element of the gripping portion 60, or it may be an element of the flexible nose cone 62, or the ring indicator may positioned between the gripping portion 60 and the flexible nose cone 62. The ring indicator 128 may also include a reflective element 129 to improve and focus the illumination effect of the light emitting source, e.g., one or more LEDs. The ring indicator 128 and the reflective element may be a single component, or more than one component (as can be seen in FIGS. 5 and 15).

Audio feedback also makes possible the feature of assisting the surgeon with monitoring nerve integrity during surgery. The insulated lead 124 connects to the operative element 110 that, in use, is positioned within the surgical field on a nerve distal to the surgical site. Stimulation of the nerve causes muscle contraction distally. The stimulation control device 22 incorporated within the housing 112 may be programmed to provide an audio tone followed by a stimulation pulse at prescribed intervals. The audio tone reminds the surgeon to observe the distal muscle contraction to confirm upon stimulation that the nerve is functioning and intact.

FIG. 15 shows an exploded view of a representative stimulation probe 50. As can be seen, the stimulation control device 22 is positioned within the housing 112. A battery 34 is electrically coupled to the control device 22. A first housing element 90 and a second housing element 92 partially encapsulate the control device 22. The ring indicator 128 and the reflective element 129 are coupled to the proximal end of the housing 112. The operative element 110 extends through the nose cone 62 and couples to the control device 22. Desirably, the stimulation probe 50 will be constructed in a manner to conform to at least the IPX1 standard for water ingress.

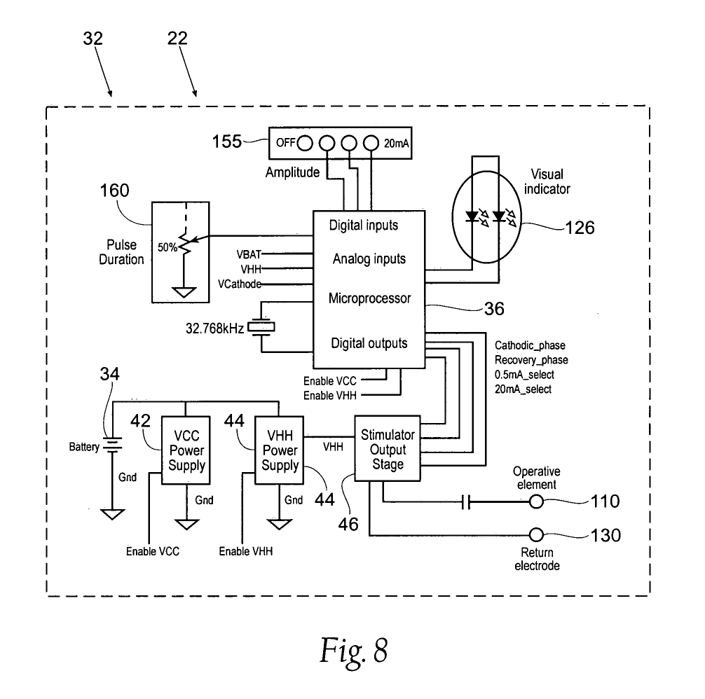

Alternatively, as FIG. 2 shows, the stimulation control device 22 may be housed in a separate case, with its own input/output (I/O) controls 26. In this alternative arrangement, the stimulation control device 22 is sized small enough to be easily removably fastened to a surgeon's arm or wrist during the surgical procedure, or otherwise positioned in close proximity to the surgical location (as shown in FIG. 7), to provide sufficient audio and/or visual feedback to the surgeon.

In this arrangement, the separate stimulation control device 22 can be temporarily coupled by a lead to a family of various medical devices for use.

The present invention includes a method of identifying/locating tissue, e.g., a nerve or muscle, in a patient that comprises the steps of providing a hand-held stimulation probe 50, 100 as set forth above, engaging a patient with the first operative element 110 and the second electrode 130, moving the power switch 155 to an activation position causing a stimulation signal 29 to be generated by the stimulation control device 22 and transmitted to the first operative element 110, through the patient's body to the second electrode 130, and back to the stimulation control device 22. The method may also include the step of observing the indicator 126 to confirm the stimulation probe 50, 100 is generating a stimulation signal. The method may also include the step of observing a tissue region to observe tissue movement or a lack thereof.

B. The Stimulation Control Device

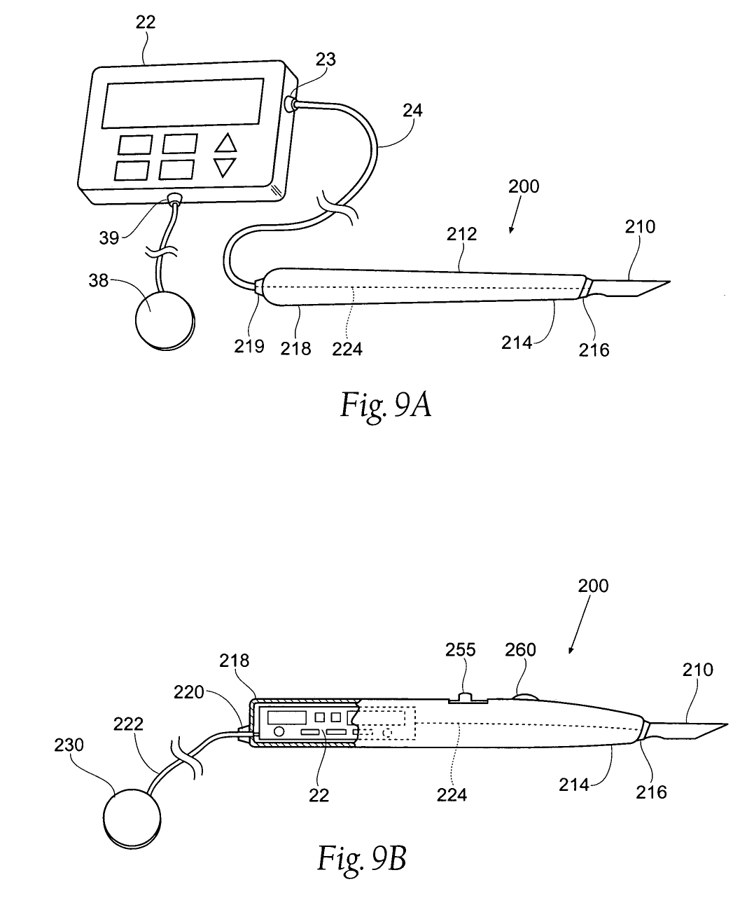

As FIG. 8 shows, the stimulation control device 22 includes a circuit 32 that generates electrical stimulation waveforms. A battery 34 desirably provides the power. The control device 22 also desirably includes an on-board, programmable microprocessor 36, which carries embedded code. The code expresses pre-programmed rules or algorithms for generating the desired electrical stimulation waveforms using the stimulus output circuit 46 and for operating the visible or audible indicator 126 based on the controls actuated by the surgeon.

In one form, the size and configuration of the stimulation control device 22 makes for an inexpensive device, which is without manual internal circuit adjustments. It is likely that the stimulation control device 22 of this type will be fabricated using automated circuit board assembly equipment and methods.

C. Incorporation with Surgical Devices

A stimulation control device 22 as just described may be electrically coupled through a lead, or embedded within various devices commonly used in surgical procedures (as previously described for the stimulation probe 50).

1. Cutting Device

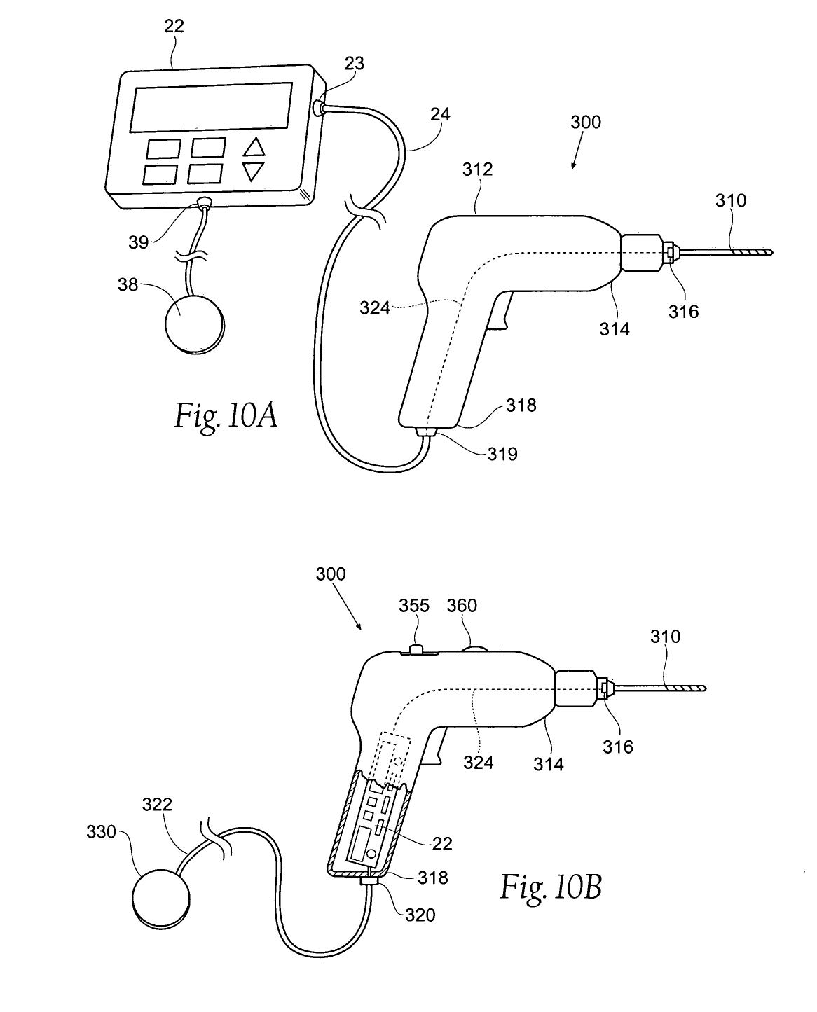

In FIGS. 9A and 9B, a device 200 is shown that incorporates all the features disclosed in the description of the stimulation probe 50, 100, except the device 200 comprises the additional feature of providing an “energized” surgical device or tool. FIG. 9A shows the tool to be a cutting device 200 (e.g., scalpel) removably coupled to a stimulation control device 22.

In the embodiment shown, the cutting device 200 includes a body 212 that carries an insulated lead 224. The insulated lead 224 connects to an operative element, such as electrode 210, positioned at the body proximal end 214 and a plug-in receptacle 219 at the body distal end 118. The lead 224 within the body 212 is insulated from the body 212 using common insulating means (e.g., wire insulation, washers, gaskets, spacers, bushings, and the like).

In this embodiment, the electrode 210 performs the cutting feature (e.g., knife or razor). The electrode 210 performs the cutting feature in electrical conductive contact with at least one muscle, or at least one nerve, or at least one muscle and nerve. The cutting device 200 desirably includes a plug-in receptacle 216 for the electrode 210, allowing for use of a variety of cutting electrode shapes and types (e.g., knife, razor, pointed, blunt, curved), depending on the specific surgical procedure being performed. In this configuration, the lead 224 electrically connects the electrode 210 to the stimulation control device 22 through plug-in receptacle 219 and lead 24.

In one embodiment, the cutting device 200 is mono-polar and is equipped with a single electrode 210 at the body proximal end 214. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the cutting device 200 may be bipolar, which precludes the use of the return electrode 38.

In the embodiment shown in FIG. 9B, the cutting device 200 accommodates within the body 212 the electrical circuitry of the stimulation control device 22. In this arrangement, the cutting device 200 may have at least two operational slide controls, 255 and 260. Power switch 255 serves a dual purpose of turning the stimulation signal to the cutting device 200 on and off, and also is stepped to control the stimulation signal amplitude selection from a predefined range (e.g., 0.5, 2.0, and 20 mA). The pulse control switch 260 allows for adjustment of the stimulation signal pulse width from a predefined range (e.g., zero through 200 microseconds).

At the body distal end 218, a second plug-in receptacle 220 may be positioned for receipt of a second lead 222. Lead 222 connects to electrode 230 which functions as a return path for the stimulation signal when the cutting device 200 is operated in a mono-polar mode.

Additionally, the device 200 may incorporate a visual or audio indicator for the surgeon, as previously described.

The present invention includes a method of identifying/locating tissue, e.g., a nerve or muscle, in a patient that comprises the steps of providing cutting device 200 as set forth above, engaging a patient with the first electrode 210 and the second electrode 230, moving the power switch 255 to an activation position causing a stimulation signal 29 to be generated by the stimulation control device 22 and transmitted to the first electrode 210, through the patient's body to the second electrode 230, and back to the stimulation control device 22. The method may also include the step of observing the indicator 126 to confirm the cutting device 200 is generating a stimulation signal. The method may also include the step of observing a tissue region to observe tissue movement or a lack thereof.

2. Drilling Device

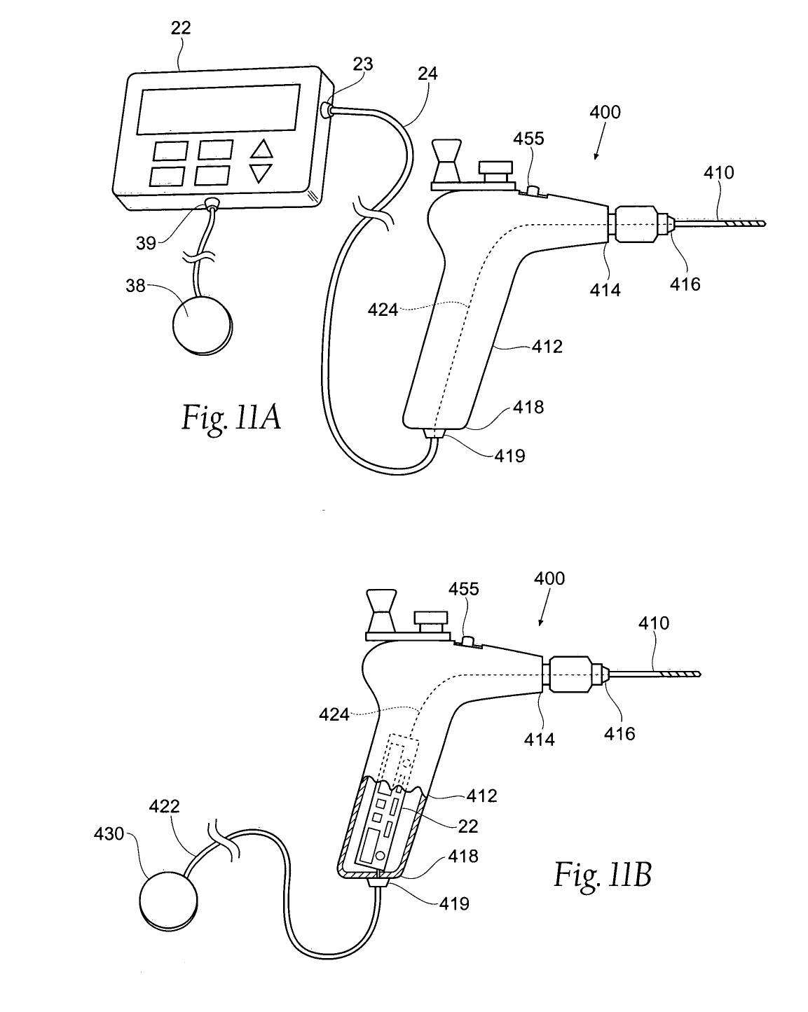

In FIGS. 10A and 10B, a device 300 is shown that incorporates all the features disclosed in the description of the stimulation probe 50, 100, except the device 300 comprises the additional feature of providing an “energized” surgical device or tool, which comprises a drilling device 300. In FIG. 10A is drilling device 300 is removably coupled to a stimulation control device 22.

In the embodiment shown, the drilling device 300 includes a body 312 that carries an insulated lead 324. The insulated lead 324 connects to an operative element, such as electrode 310, positioned at the body proximal end 314 and a plug-in receptacle 319 at the body distal end 318. The lead 324 within the body 312 is insulated from the body 312 using common insulating means (e.g., wire insulation, washers, gaskets, spacers, bushings, and the like).

In this embodiment, the electrode 310 performs the drilling feature. The electrode 310 may also perform a screwing feature as well. The electrode 310 performs the drilling feature in electrical conductive contact with a hard structure (e.g., bone).

The drilling device 300 desirably includes a plug-in receptacle or chuck 316 for the electrode 310, allowing for use of a variety of drilling and screwing electrode shapes and sizes (e.g., ¼ and ⅜ inch drill bits, Phillips and flat slot screw drivers), depending on the specific surgical procedure being performed. In this configuration, the lead 324 electrically connects the electrode 310 to the stimulation control device 22 through plug-in receptacle 319 and lead 324.

In one embodiment, the drilling device 300 is mono-polar and is equipped with a single electrode 310 at the body proximal end 314. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the drilling device 300 may be bipolar, which precludes the use of the return electrode 38.

In FIG. 10B, the drilling device 300 is shown to accommodate within the body 312 the electrical circuitry of the stimulation control device 22. The drilling device 300 may have at least two operational slide controls, 355 and 360. Power switch 355 serves a dual purpose of turning the stimulation signal to the drilling device 300 on and off, and also is also stepped to control the stimulation signal amplitude selection from a predefined range (e.g., 0.5, 2.0, and 20 mA). The pulse control switch 360 allows for adjustment of the stimulation signal pulse width from a predefined range (e.g., zero through 200 microseconds). At the body distal end 318, a second plug-in receptacle 320 may be positioned for receipt of a second lead 322. Lead 322 connects to electrode 330 which functions as a return path for the stimulation signal when the drilling device 300 is operated in a mono-polar mode.

Additionally, the device 300 may incorporate a visual or audio indicator for the surgeon, as previously described.

The present invention includes a method of identifying/locating tissue, e.g., a nerve or muscle, in a patient that comprises the steps of providing a drilling device 300 as set forth above, engaging a patient with the first electrode 310 and the second electrode 330, moving the power switch 355 to an activation position causing a stimulation signal 29 to be generated by the stimulation control device 22 and transmitted to the first electrode 310, through the patient's body to the second electrode 330, and back to the stimulation control device 22. The method may also include the step of observing the indicator 126 to confirm the drilling device 400 is generating a stimulation signal. The method may also include the step of observing a tissue region to observe tissue movement or a lack thereof.

3. Pilot Auger

An additional aspect of the invention provides systems and methods for controlling operation of a family of stimulating devices comprising a stimulation control device electrically coupled to a pilot auger for hard surface rotary probing.

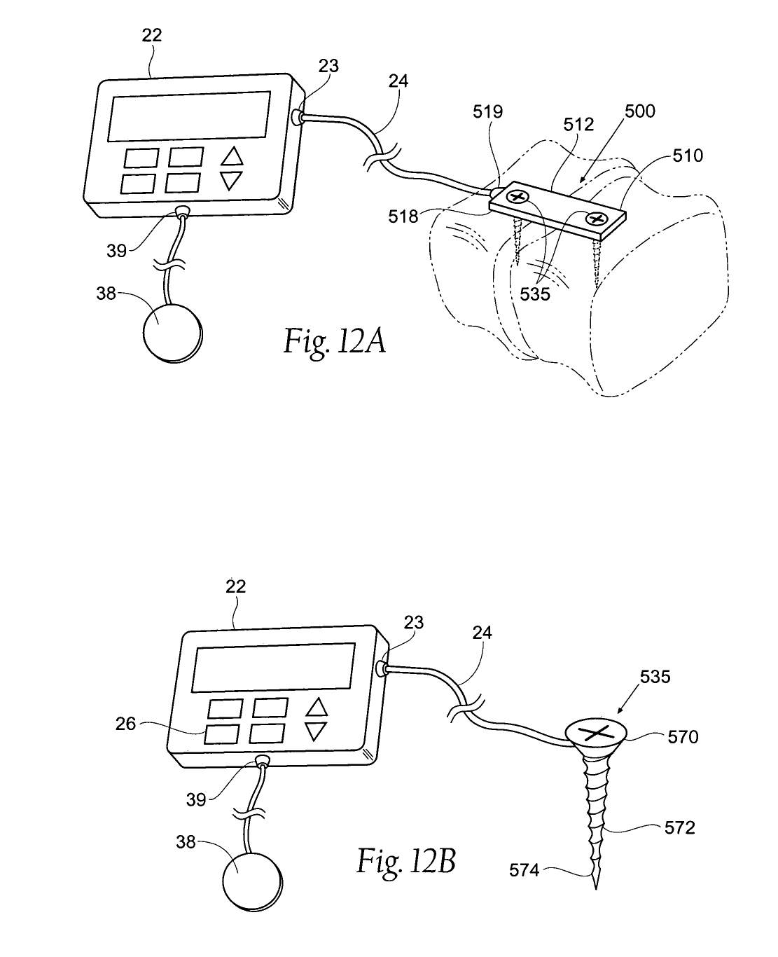

This embodiment incorporates all the features disclosed in the description of the stimulation probe 50, 100, except this embodiment comprises the additional feature of providing an “energized” surgical device or tool. FIG. 11A shows a pilot auger device 400 removably coupled to a stimulation control device 22. In the embodiment shown, the pilot auger device 400 includes a body 412 that carries an insulated lead 424. The insulated lead 424 connects to an operative element, such as an electrode 410, positioned at the body proximal end 414 and a plug-in receptacle 419 at the body distal end 418. The lead 424 within the body 412 is insulated from the body 412 using common insulating means (e.g., wire insulation, washers, gaskets, spacers, bushings, and the like). In this embodiment, the electrode 410 performs the pilot augering feature. The electrode 410 performs the pilot augering feature in electrical conductive contact with a hard structure (e.g., bone).

The pilot auger device 400 desirably includes a plug-in receptacle or chuck 416 for the electrode 410, allowing for use of a variety of pilot augering electrode shapes and sizes (e.g., 1/32, 1/16, and ⅛ inch), depending on the specific surgical procedure being performed. In this configuration, the lead 24 electrically connects the electrode 410 to the stimulation control device 22 through plug-in receptacle 419 and lead 24.

In one embodiment, the pilot auger device 400 is mono-polar and is equipped with a single electrode 410 at the body proximal end 414. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the pilot auger device 400 may be bipolar, which precludes the use of the return electrode 38.

As FIG. 11B shows. the pilot auger device 400 may accommodate within the body 412 the electrical circuitry of the stimulation control device 22. At the body distal end 418, a second plug-in receptacle 420 may be positioned for receipt of a second lead 422. Lead 422 connects to electrode 430 which functions as a return path for the stimulation signal when the pilot auger device 400 is operated in a mono-polar mode.

The pilot auger device 400 includes a power switch 455. When moved to an activation position, a stimulation signal is generated by the stimulation control device 22. Additionally, the device 400 may incorporate a visual or audio indicator for the surgeon, as previously described.

The present invention includes a method of identifying/locating tissue, e.g., a nerve or muscle, in a patient that comprises the steps of providing a pilot auger device 400 as set forth above, engaging a patient with the first electrode 410 and the second electrode 430, moving the power switch 455 to an activation position causing a stimulation signal to be generated by the stimulation control device 22 and transmitted to the first electrode 410, through the patient's body to the second electrode 430, and back to the stimulation control device 22. The method may also include the step of observing the indicator 126 to confirm the pilot auger device 400 is generating a stimulation signal. The method may also include the step of observing a tissue region to observe tissue movement or a lack thereof.

D. Incorporation with Fixation Devices

An additional aspect of the invention provides systems and methods for controlling operation of a family of stimulating devices comprising a stimulation control device electrically coupled to a fixation device or a wrench or screwdriver for placing the fixation device. A fixation device (e.g., orthopedic hardware, pedicle screws) is commonly used during spinal stabilization procedures (fusion), and internal bone fixation procedures.

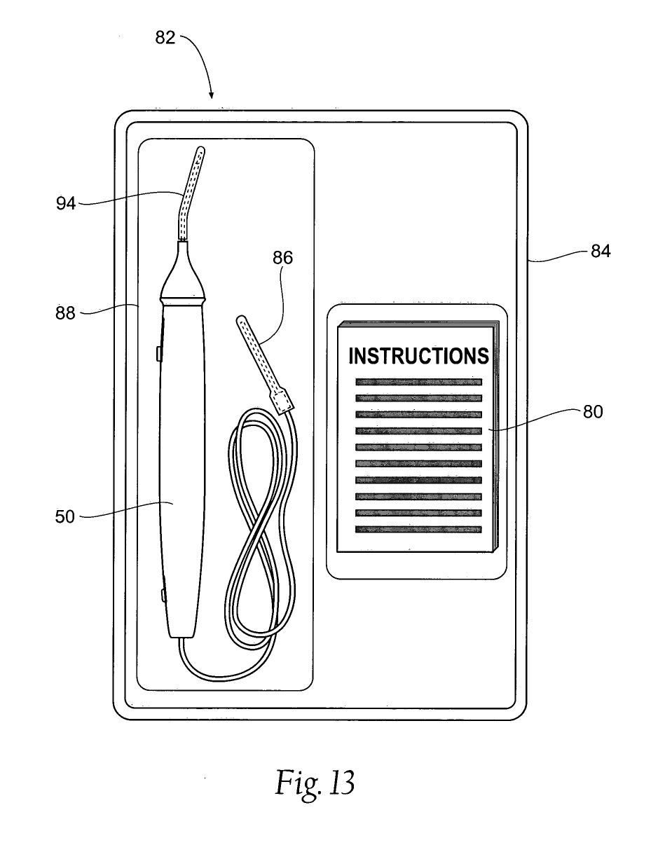

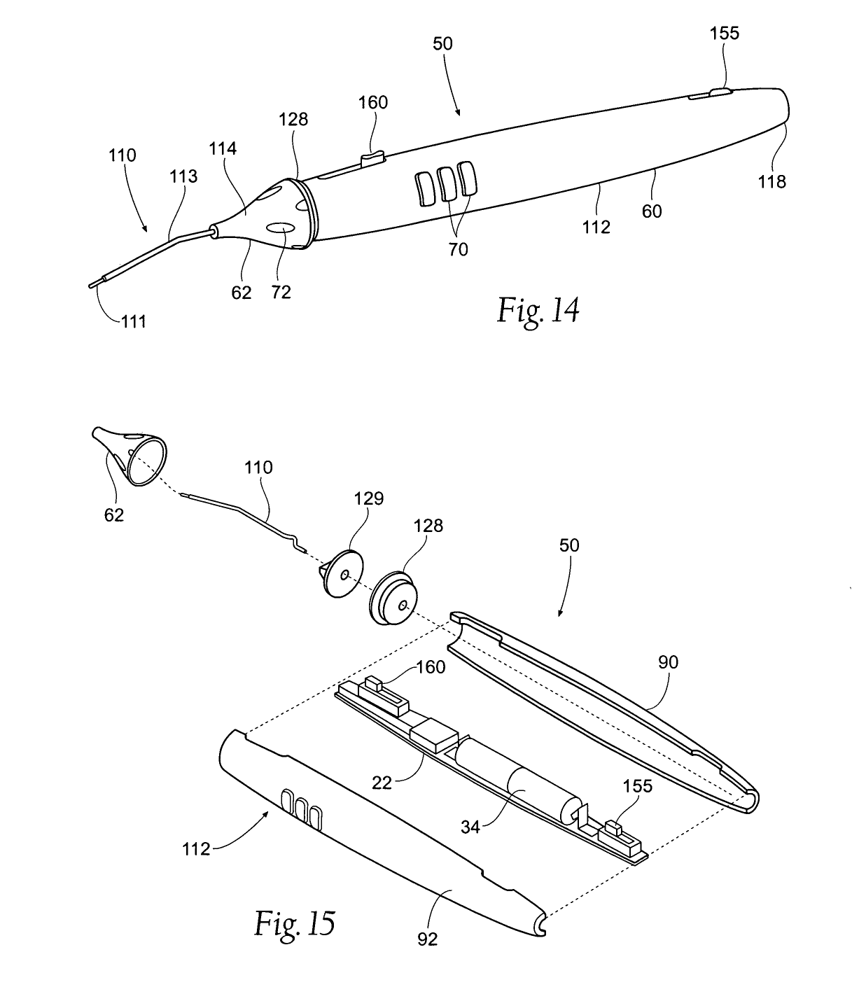

This embodiment incorporates all the features disclosed in the description of the stimulation probe 50, 100, except this embodiment comprises the additional feature of providing an “energized” fixation device or tool. FIG. 12A shows a fixation device 500 removably coupled to a stimulation control device 22. In the embodiment shown, the fixation device 500 includes a rectangularly shaped body 512 that also serves as an operative element, such as electrode 510. The fixation device 500 may take on an unlimited number of shapes as necessary for the particular procedure taking place. Pedicle screws 535 may be used to secure the fixation device to the bony structure. The electrode 510 performs the fixation feature in electrical conductive contact with a hard structure (e.g., bone).

The fixation device 500 or wrench or screwdriver for placing the fixation device desirably includes a plug-in receptacle 519. The fixation device 500 may take on an unlimited variety of shapes and sizes depending on the specific surgical procedure being performed. In this configuration, the lead 24 electrically connects the electrode 510 to the stimulation control device 22 through plug-in receptacle 519.

In one embodiment, the fixation device 500 is mono-polar and is equipped with the single electrode 510. In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. The return electrode 38 may be attached to the stimulation device 22 by way of a connector or plug-in receptacle 39. In an alternative embodiment, the fixation device 500 may be bipolar, which precludes the use of the return electrode 38.

In yet an additional alternative embodiment (see FIG. 12B), the fixation device may be a pedicle screw 535. The pedicle screw 535 is removably coupled to a stimulation control device 22. In the embodiment shown, the pedicle screw 535 includes a head 570 and a shaft 572, which both serve as an operative element, such as electrode 574. The electrode 574 performs the fixation feature in electrical conductive contact with a hard structure (e.g., bone), as the pedicle screw 535 is being positioned within a bony structure. The lead 24 electrically connects the electrode 574 to the stimulation control device 22, through a break-away connection or other similar electrical connective means. The fixation device 535 may take on an unlimited variety of shapes and sizes depending on the specific surgical procedure being performed.

In the mono-polar mode, the stimulation control device 22 includes a return electrode 38 which functions as a return path for the stimulation signal. Electrode 38 may be any of a variety of electrode types (e.g., paddle, needle, wire, or surface), depending on the surgical procedure being performed. In an alternative embodiment, the fixation device 500 may be bipolar, which precludes the use of the return electrode 38.

The present invention includes a method of identifying/locating tissue, e.g., a nerve or muscle, in a patient that comprises the steps of providing a fixation device 500 as set forth above, engaging a patient with the first electrode 510 and the second electrode 38, turning power on to the stimulation control device 22 through the I/O controls 26, causing a stimulation signal 29 to be generated by the stimulation control device 22 and transmitted to the first electrode 510, through the patient's body to the second electrode 38, and back to the stimulation control device 22. The method may also include the step of observing the indicator 126 to confirm the fixation device 500 is generating a stimulation signal. The method may also include the step of observing a tissue region to observe tissue movement or a lack thereof.

### IV. Technical Features

The stimulation control device 22, either alone or when incorporated into a stimulation probe or surgical device, can incorporate various technical features to enhance its universality.

A. Small Size

According to one desirable technical feature, the stimulation control device 22 can be sized small enough to be held and used by one hand during surgical procedures, or to be installed within a stimulation probe or surgical device. The angle of the stimulating tip facilitates access to deep as well as superficial structures without the need for a large incision. Visual and/or audible indication incorporated in the housing provides reliable feedback or status to the surgeon as to the request and delivery of stimulus current.

According to an alternative desirable technical feature, the stimulation control device 22 may also be sized small enough to be easily removably fastened to a surgeon's arm or wrist during the surgical procedure, or positioned in close proximity to the surgical location (as shown in FIG. 7), to provide sufficient audio and/or visual feedback to the surgeon.

B. Power Source

According to one desirable technical feature, power is provided by one or more primary batteries 34 for single use positioned inside the housing and coupled to the control device 22. A representative battery 34 may include a size “N” alkaline battery. In one embodiment, two size “N” alkaline batteries in series are included to provide a 3 volt power source. This configuration is sized and configured to provide an operating life of at least seven hours of operation—either continuous or intermittent stimulation.

C. The Microprocessor/Microcontroller

According to one desirable technical feature, the stimulation control device 22 desirably uses a standard, commercially available micro-power, flash programmable microcontroller 36. The microcontroller 36 reads the controls operated by the surgeon, controls the timing of the stimulus pulses, and controls the feedback to the user about the status of the instrument (e.g., an LED with 1, 2, or more colors that can be on, off, or flashing).

The microcontroller operates at a low voltage and low power. The microcontroller send low voltage pulses to the stimulus output stage 46 that converts these low voltage signals into the higher voltage, controlled voltage, or controlled current, stimulus pulses that are applied to the electrode circuit. This stimulus output stage 46 usually involves the use of a series capacitor to prevent the presence of DC current flow in the electrode circuit in normal operation or in the event of an electronic component failure.

### V. Representative Use of a Stimulation Probe

The stimulation probe 50, 100, as described, make possible the application of a stimulation signal at sufficiently high levels for the purposes of locating, stimulating, and evaluating nerve or muscle, or both nerve and muscle integrity in numerous medical procedures, including, but not limited to, evaluating proximity to a targeted tissue region, evaluating proximity to a nerve or to identify nerve tissue, evaluating if a nerve is intact (i.e., following a traumatic injury) to determine if a repair may be needed, evaluating muscle contraction to determine whether or not the muscle is innervated and/or whether the muscle is intact and/or whether the muscle is severed, and evaluating muscle and tendon length and function following a repair or tendon transfer prior to completing a surgical procedure.

Instructions for use 80 are desirably included in a kit 82 along with a stimulation probe 50. The kit 82 can take various forms. In the illustrated embodiment, kit 82 comprises a sterile, wrapped assembly. A representative kit 82 includes an interior tray 84 made, e.g., from die cut cardboard, plastic sheet, or thermo-formed plastic material, which hold the contents. Kit 82 also desirably includes instructions for use 80 for using the contents of the kit to carry out a desired therapeutic and/or diagnostic objectives.

The instructions 80 guide the user through the steps of unpacking the stimulation probe 50, positioning the electrodes, and disposing of the single use disposable stimulator 50. Representative instructions may include, but are not limited to:
Remove the stimulation probe 50 from sterile package 88.
Remove cover 94 (e.g., a silicone cover) from the operative element 110.
Remove protective cover 86 from the return electrode 131.
Position the return electrode 131 in contact with the patient such that:
   1. The return electrode is desirably positioned in an area remote from the area to be stimulated.
   2. The return electrode is desirably not positioned across the body from the side being stimulated.
   3. The return electrode is desirably not in muscle tissue.
Turn the stimulation probe 500N by moving the power switch 155 from OFF to the 0.5 mA setting (or greater). The stimulation probe 50 desirably is turned ON before the operative element 110 makes contact with tissue.
The indicator 126 will be illuminated yellow (for example) continuously if the stimulation probe 50 is ON, but not in contact with tissue.
Contact tissue with the operative element 110.
Adjust the pulse control 160 gradually to increase the level of stimulation. The indicator 126 will flash yellow indicating that stimulation is being delivered.
A flashing red (for example) indicator 126 means that stimulation has been requested, but no stimulation is being delivered because of inadequate connection of the operative element 110 or the return electrode 131 to the patient tissue. Check the return electrode contact and position, and check the operative element 110 contact and position.
Placing the power switch 155 to the off/standby position will stop stimulation and the visual indictor 126 will be illuminated yellow continuously.
Placing the pulse control 160 at the minimum position will stop stimulation and the visual indictor 126 will be illuminated yellow continuously.
A low/depleted battery 34 will cause the stimulation probe 50 to automatically turn OFF and the visual indicator 126 will not be illuminated. No further use of the stimulator 50 will be possible.
At end of use, move the power switch 155 to the off/standby position and move the pulse control 160 to the minimum position.
Cut off and dispose of the return electrode 131 in an appropriate sharps/biohazard container.
Dispose of the stimulation probe 50 per hospital or facility guidelines.

Nerve location may be performed for a variety of reasons, including location for identification prior to or during a “nerve cleaning” procedure, and location for avoidance of iatrogenic nerve injury. One problem that has long persisted in the field of reconstructive and microvascular surgery is the continued occurrence of iatrogenic nerve injury during surgery. Iatrogenic nerve injury during surgery is a deservedly feared complication, resulting in pain and possible permanent loss of function for the patient and malpractice litigation and probable liability for the physician. One retrospective study of 444 randomly sampled malpractice claims revealed that 14% were peripheral nerve injuries.

Reported nerve injury rates are surprisingly high and, as with most complications, are probably underreported.

While nerve injury is perhaps not totally avoidable, the capacity to stimulate nerves and muscles intraoperatively makes surgery safer and more predictable, and improves outcomes. This is difficult when operating through areas scarred by trauma, infection, tumors or previous surgery that obliterates the normal, anatomical landmarks. Distinguishing nerve from adjacent tissue is difficult or impossible by visual inspection. The ability to electrically stimulate tissue to elicit a response is frequently of crucial importance to identify and protect nerves whose identity and location are obscured by scarring and abnormal anatomy.

While various nerve stimulators exist, they have been problematic for a number of reasons. For instance, scar is an effective insulator and it has been discovered that a lack of response to electrical stimulation from existing nerve stimulators may be due to inadequate stimulus intensity. Thus, failure to elicit a response with conventional intraoperative stimulation may have indicated that the structure in question was not a nerve and was, therefore, safe to cut. However, it may also have meant that the stimulator was not functioning properly or that the stimulus provided by the prior art stimulator was insufficient to stimulate the nerve due to, e.g., scar-related, or other, dysfunction. When there is a failure to elicit a response, and a surgeon is still suspicious, the surgeon must extend the surgical exposure time significantly or call for the operating microscope to dissect around the structure in question thought to be innervated by nerve tissue. These processes may take considerable time, will add to the service cost through extra operating room time and expensive billing codes for microsurgery, and are not processes that most orthopedic surgeons can actually perform. Existing stimulators have been unreliable and undependable (the Vari-Stim® by Medtronic Xomed, Inc., has been recalled; Recall # Z-0947-2009), which adds to the problem of uncertainty.

Alternatively, rather than locating a nerve and then performing a surgical procedure remotely from the located nerve, it may be desirable to locate a nerve precisely to perform a surgical procedure adjacent to or on the nerve tissue. Very broad, but safe, stimulation capability, from stimulating entire muscle regions to individual nerve fibers, is desirable. These features enable the surgeon to avoid dangerous “false negative” responses, and allow the surgeon to perform threshold testing in a semi-quantitative manner. Indeed, wide-range, continuously-variable stimulus capability allows the surgeon to hone in on an area suspected of containing nerve tissue and then precisely localizing the nerve by beginning with a higher stimulus intensity and gradually lowering the intensity as the nerve is excavated from the scar tissue. This has allowed identification of nerves that were heavily scarred and indistinguishable from adjacent tissue and, in several cases, has avoided erroneous sacrifice of a critical nerve that would have significantly, detrimentally, affected the outcome of a surgery.

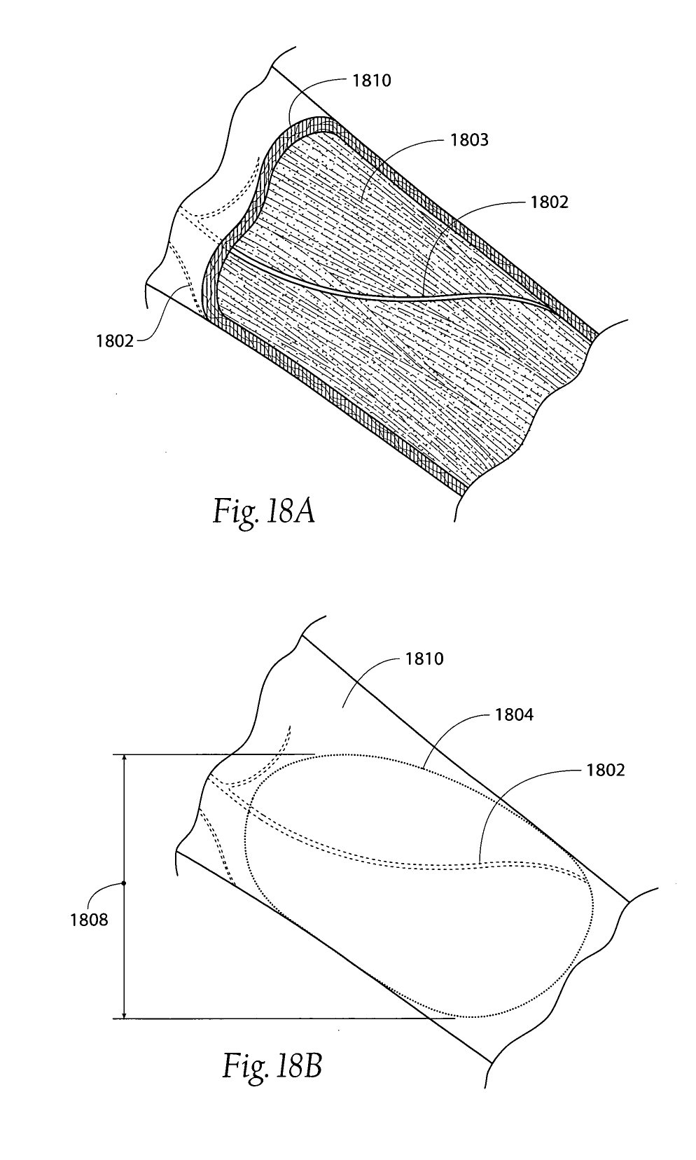

A first embodiment 1600 of a method according to the present invention includes the steps depicted in FIG. 16. In one step or series of steps 1602, a first potential incision location and a first potential incision length are determined and/or identified. A first electrical stimulation is applied 1604 to a tissue region located along at least a portion of the first potential incision length. During and in response to the first electrical stimulation 1604, a neural response may or may not be observed 1606. For example, where nerve fibers are activated by the first electrical stimulation, and such nerve fibers innervate muscle, the muscle may twitch and/or fully contract. For instance, sufficient stimulation of the axillary nerve will likely cause contraction of the deltoid muscle in a human shoulder, which can be visibly observed. Arthroscopically, stimulation may be visibly observed as a jump or movement of the innervated tissue within the field of an arthroscope. If a neural response is observed, an alternate potential incision location and associated desired incision length may be determined and/or identified 1608. The operative element 110 of a stimulator 50 may be relocated 1610 to contact a second tissue region located along the alternate desired incision length. A stimulation may again be applied 1604, this time to at least a portion of the second tissue region. Steps of this method may be repeated at a predetermined or desired stimulation level until no neural response is observed at a final incision location. Once no neural response to the subsequent stimulation(s) is observed, and if the surgeon is comfortable with the intensity of the electrical stimulation applied, the surgeon may be confident in incising the final incision location 1612.

The method may further include the steps of altering electrical stimulation parameters, such as by increasing or decreasing the electrical stimulation pulse current amplitude and/or the pulse time duration. Such altering of stimulation parameters may occur, for example, after it has been determined whether a neural response was generated by previous stimulation. For instance, it may be desirable to confirm the neural response to determine whether the observed response or lacking response were false. Such confirmation may be made by adjusting stimulation parameters 1614,1616 and then again applying a stimulation 1604 to the same tissue region that was stimulated when the neural response determination 1606 was made. For example, if no neural response was observed, it may be desirable to adjust electrical stimulation parameters 1614 to increase stimulation intensity (pulse duration and/or amplitude) of the electrical stimulation to confirm that the lacking neural response was not a false negative. Additionally, if a neural response was observed, it may be desirable to adjust electrical stimulation parameters 1616 to decrease stimulation intensity (pulse duration and/or amplitude) to confirm that the neural response was not a false positive. The stimulation pulse train is preferably provided continuously once it has started, but it may optionally be paused or stopped.

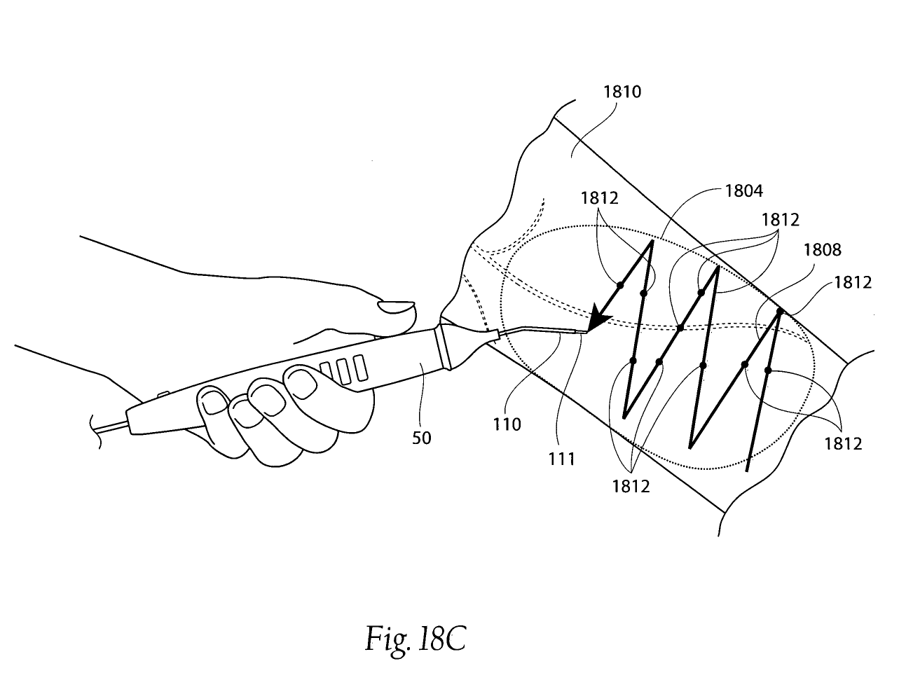

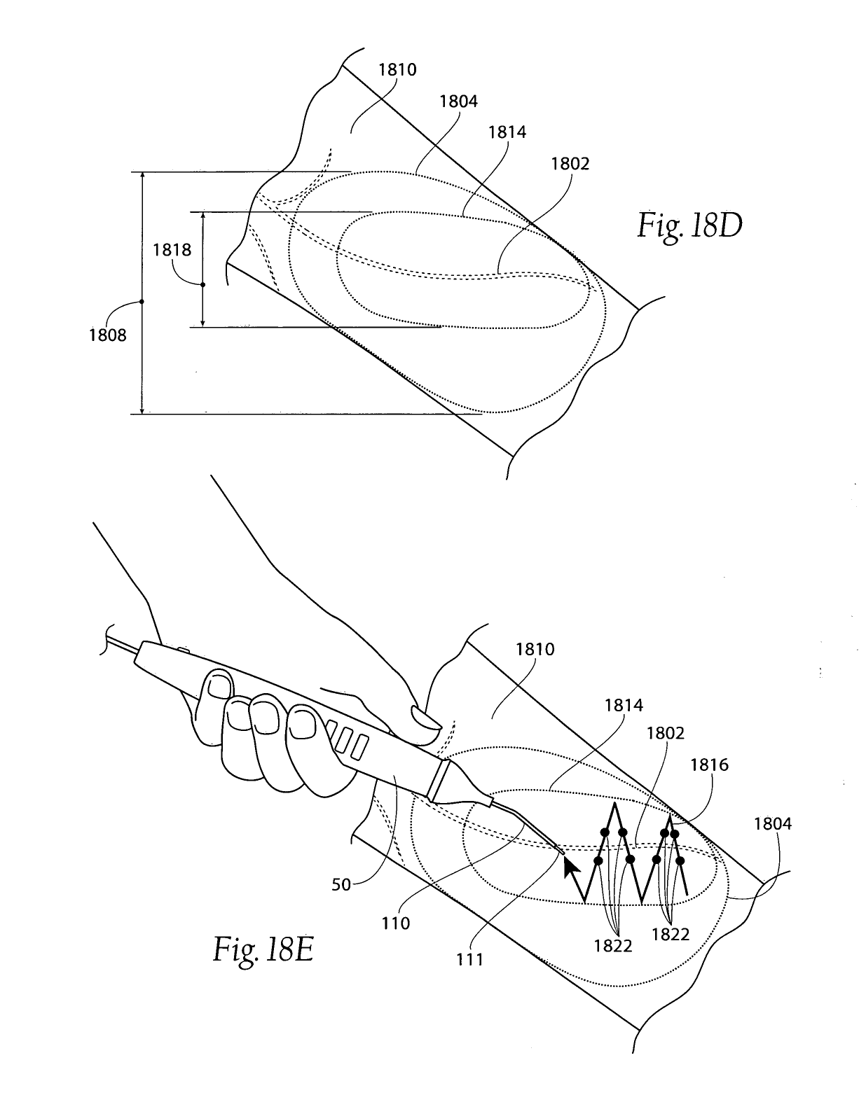

A second embodiment 1700 of a method according to the present invention is shown in FIG. 17. Reference may also be had to FIGS. 18A-18F to aid in understanding the following description. The second method embodiment 1700 generally includes a method for honing in on a precise location of one or more nerve fibers 1802, perhaps not for the purpose of avoiding fibers 1802, but for the purpose of operating directly on one or more of the fibers 1802. The nerve fibers 1802 may innervate muscle tissue 1803, which may be disposed beneath a tissue layer 1810 to be stimulated. In one step 1702, a target stimulation tissue region 1804 is identified. In that tissue region 1804, using an operative element 110 of a handheld stimulator 50, a first electrical stimulation is applied 1704 along a first stimulation path 1806, across a first tissue stimulation width 1808. Preferably, this first electrical stimulation may be provided at a maximum stimulation intensity to be used during the prospecting or honing method, such as an amplitude of 20 mA and a pulse duration of greater than zero and less than or equal to 200 μs. The first electrical stimulation may also be provided at a minimal stimulation intensity and increased until some neural response is detected, and then the level could be altered from that point to be further increased, but preferably decreased. Furthermore, this first electrical stimulation may be provided while translating the electrode 111 along the stimulation path 1808 while in contact with tissue 1810 in the tissue region 1804. In response to the applied electrical stimulation applied at various stimulation locations, a neural response may be generated and observed. If no response is generated and/or observed, the electrode 111 or operative element 110 of the stimulator 50 may be relocated 1706 to a different position, within or without the stimulation region 1804, and a subsequent stimulation 1704 applied. If a neural response is generated and/or observed, the surgeon may identify, measure, and/or document 1708 one or more active stimulation locations 1812 at which such a response is observed. In other words, one or more positions of the electrode 111, at which a neural response is generated in response to an electrical stimulation, is or are identified, measured, and/or documented 1708.

Since the first stimulation is provided at a maximum stimulation level, an active region 1814 including active stimulation locations 1812 is likely to be identified for a given nerve or set of nerve fibers 1802. This active region 1814 is likely to be smaller in size in at least one dimension than the tissue region 1804 identified to be at least partially stimulated prior. That is, a tissue region 1804 is swept to identify a smaller, or focused, active region 1814. The sweeping of a stimulation path may be done continuously, or interruptedly, and may be of any pattern, though a zig-zag or spiral pattern is preferred. Once an active region 1814 is identified, which may include one or more of the identified active stimulation locations 1812, a parameter of the electrical stimulation to be applied is modified or adjusted 1710. For instance, the stimulation intensity (pulse duration and/or amplitude) could be reduced. After the modification of the stimulation parameter 1710, then a second electrical stimulation 1704 is applied to the tissue region 1804. The second stimulation 1704 may be confined to the active region 1814 within the tissue region 1804, or may extend beyond the active region 1814. Preferably, the second stimulation 1704 is confined to a second stimulation path 1816 located entirely within the previously identified active region 1814 so as to minimize the stimulation area and reduce the time in which a nerve is located. In response to the second electrical stimulation 1704 applied at various stimulation locations, preferably all contained within the previously identified active region 1814, a second neural response may be generated and observed. The second neural response may be generated by stimulation of the same nerve or nerve fibers 1802 that were stimulated by the first stimulation. The surgeon may identify, measure, and/or document 1708 one or more active stimulation locations 1822 at which such response is observed. In other words, one or more positions of the electrode 111, at which a neural response is generated in response to the second stimulation 1704, is or are identified, measured, and/or documented.

The process of modifying at least one stimulation parameter and then applying stimulation may be repeated as many times as desired to achieve an active region of a desired size, indicative of present neural fibers. In such iterative application, a previously identified active region preferably becomes the next stimulation region, such that with each iteration, the area of tissue 1810 to be stimulated decreases. If the active region is of a desired size or a given stimulation intensity has been reached, thereby possibly limiting the narrowness of the active region, the method may be ended having identified an active region of a desired size or the method may continue with, for example, an incision that may be made 1712 near the active region in an attempt to, for example, expose the nerve or nerve fibers 1802.

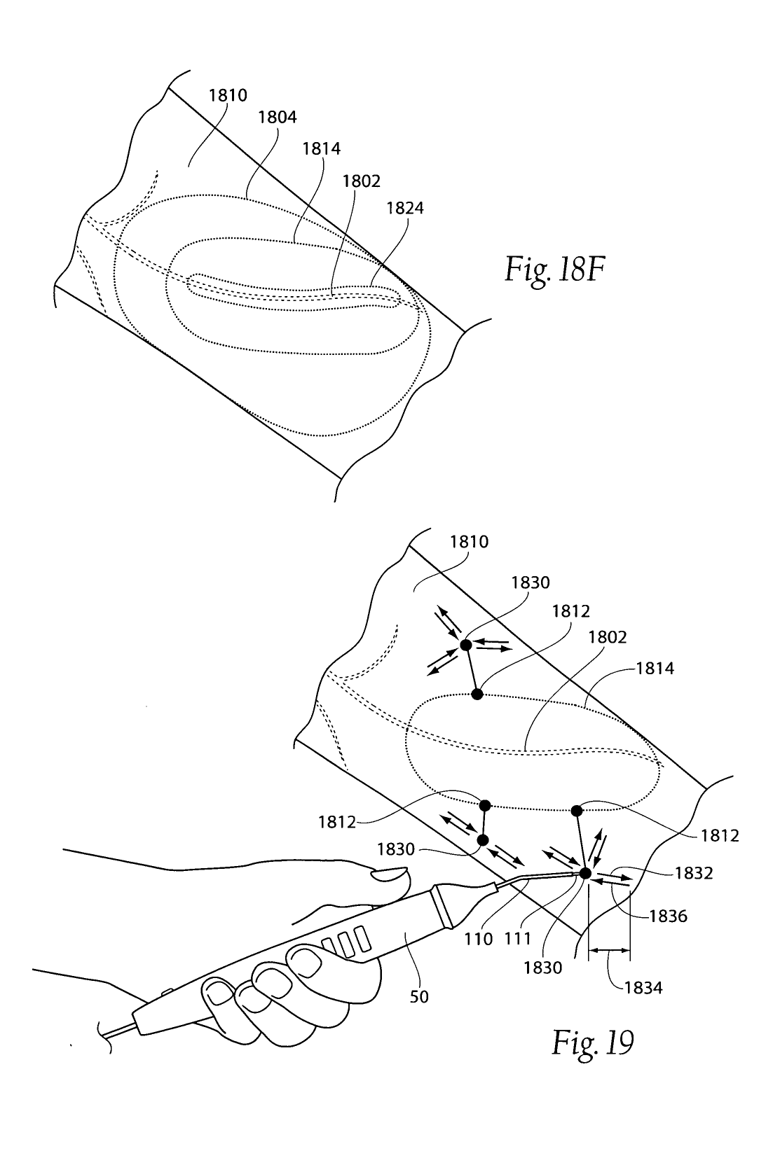

In a variation of the second embodiment 1700, the identification step 1702, in which a stimulation region 1804 is identified, may be eliminated and replaced by, or supplemented with, a prospecting step. Reference to FIGS. 17 and 19 may assist in understanding the following explanation. For instance, a surgeon may be completely unaware of a path or paths of nerve fibers 1802 that run beneath or within a tissue 1810 to be stimulated. To determine a desired stimulation region 1804 or active region 1814, one or more prospecting points 1830 located on or in the tissue 1810 may be selected. An electrical stimulation is applied at one of the prospecting points 1830 using an electrode 111 disposed on an operative element 110 of a stimulation probe 50. Preferably while in contact with the tissue 1810, the electrode ill may be moved in a desired pattern to attempt to identify active stimulation locations 1812. While zig-zag and spiral patterns have already been mentioned, when prospecting a star pattern may prove beneficial. Such pattern may be traced by the electrode 111 in contact with the tissue 1810 in the following manner. Electrical stimulation may be applied at a prospecting location 1830 to determine whether neural response is generated. If no neural response is detected, the electrode 111, preferably while in contact with the tissue 1810, may be translated radially outwardly in a first direction 1832 for a first honing distance 1834. If a neural response is detected, an active stimulation location 1812 may be identified or noted. If no neural response is detected, the electrode 111, preferably while in contact with the tissue 1810, may be translated radially inwardly in a second direction 1836 opposite the first direction 1832 through the first honing distance 1834, back to the prospecting location 1830. The radially outward translation can then be repeated until at least one active stimulation location 1812 is determined. Subsequent honing translations may be done at through different honing distances 1834 and in different directions, though similar or the same honing distances 1834 are preferred from a given prospecting location. While more than one active stimulation location 1812 may be determined from the process originating at a single prospecting location 1830, it is preferred that once a first active stimulation location 1812 is located, the electrode 111 may be disengaged from the tissue 1810, and moved to a subsequent prospecting location 1830, to continue the prospecting process. After one or more active stimulation locations 1812 have been identified, thereby establishing an active region 1814, the method embodiment 1700 may then proceed as desired, such as by reducing the stimulation intensity 1708, and so on.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method of avoiding nerve tissue in a surgical procedure, the method comprising the steps of:
identifying a first potential incision location and a first potential incision length;
in a first application step, applying a first electrical stimulation, with an electrode, to a first tissue region along the first potential incision length;
during the first application step, observing a neural response to the first stimulation;
identifying a second incision location and a second potential incision length;
in a second application step, applying a second electrical stimulation, with the electrode, to a second tissue region along the second potential incision length;
during the second application step, observing no neural response to the second stimulation; and
after observing no neural response to the second stimulation, performing a surgical procedure through the second tissue region.

2. A method according to claim 1, wherein the step of performing a surgical procedure comprises the step of incising a portion of the second tissue region along a portion of the second potential incision length.

3. A method according to claim 1, further comprising, after observing the neural response to the first stimulation, the steps of:
adjusting a parameter of the first electrical stimulation; and
in a third application step, applying a third electrical stimulation, with an electrode, to the first tissue region along the first potential incision length.

4. A method according to claim 3, wherein the adjusting step comprises the step of decreasing stimulation intensity of the first electrical stimulation by reducing one of electrical stimulation pulse duration and electrical stimulation amplitude.

5. A method according to claim 1, further comprising, after observing no neural response to the second stimulation, the steps of:
adjusting a parameter of the second electrical stimulation; and
in a third application step, applying a third electrical stimulation, with an electrode, to the second tissue region along the second potential incision length.

6. A method according to claim 5, wherein the adjusting step comprises the step of increasing stimulation intensity of the second electrical stimulation by increasing one of electrical stimulation pulse duration and electrical stimulation amplitude.

7. A method of locating nerve tissue to perform a surgical procedure, the method comprising the steps of:
identifying a first potential incision location and a first potential incision length;
in a first application step, applying a first electrical stimulation, with an electrode, to a tissue region along the first potential incision length;
during the first application step, observing no desired neural response to the first stimulation;
altering a parameter of the electrical stimulation;
in a second application step, applying a second stimulation to the tissue region;
during the second application step, observing a desired neural response to the second stimulation;
after the first and second application steps, making an incision along the first potential incision length;
performing a surgical procedure through the incision;
in a third application step, applying the first stimulation to the tissue region, observing the desired neural response during the third application step.

8. A method according to claim 7, wherein the first application step is performed before the altering step and the second application step is performed after the altering step.

9. A method according to claim 7, wherein the altering step comprises the step of increasing electrical stimulation intensity.

10. A method according to claim 9, wherein the step of increasing electrical stimulation intensity comprises the step of increasing at least one of electrical stimulation amplitude and electrical stimulation pulse duration.

11. A method according to claim 7, wherein the first application step further comprises the step of translating the electrode along at least a portion of the first incision length while the electrode is in contact with the tissue.

12. A method according to claim 11, wherein the first electrical stimulation is applied during the translating step.

13. A method according to claim 7, wherein the surgical procedure comprises the step of removing scar tissue through the incision.

14. A method according to claim 1, wherein the electrode is coupled to a first device and a second device is used for the surgical procedure.

15. A method according to claim 14, wherein the second device is a scalpel.

16. A method for honing in on nerve fibers disposed below or innervating animal tissue, the method comprising the steps of:
at a first stimulation intensity, applying a first electrical stimulation to animal tissue within an identified stimulation region, the first electrical stimulation being applied with an electrode in contact with the tissue;
in a first identifying step, identifying a plurality of first active stimulation locations within the stimulation region at which one or more neural response is generated in response to the first electrical stimulation, the first active stimulation locations being disposed about a perimeter of a focused stimulation region;
at a reduced stimulation intensity, applying a second electrical stimulation within the focused stimulation region; and
in a second identifying step, identifying a second active stimulation location within the focused stimulation region at which one or more neural response is generated in response to the second electrical stimulation.

17. A method according to claim 16, wherein the first identifying step comprises the step of translating the electrode across the tissue while applying the first electrical stimulation.

18. A method according to claim 17, wherein the translating step comprises the step of translating the electrode in a desired pattern.

19. A method according to claim 18, wherein the pattern comprises a star pattern.

20. A method according to claim 18, wherein the pattern comprises a spiral pattern.

21. A method according to claim 16, wherein the second identifying step comprises the step of translating the electrode across the tissue while applying the second electrical stimulation.

22. A method according to claim 21, wherein the translating step comprises the step of translating the electrode in a desired pattern.

23. A method according to claim 22, wherein the pattern comprises a star pattern.

24. A method according to claim 22, wherein the pattern comprises a spiral pattern.

25. A method according to claim 16, wherein the reduced stimulation intensity is generated by a step of altering an electrical stimulation parameter of the first electrical stimulation.

26. A method according to claim 25, wherein the step of altering comprises reducing one of electrical pulse stimulation pulse duration and electrical stimulation amplitude.

27. A method of seeking a neural response to electrical stimulation, the method comprising the steps of:
identifying a prospecting location on animal tissue;
applying electrical stimulation to the tissue at the prospecting location with an electrode to determine whether a neural response is generated;
observing no neural response to the electrical stimulation, translating the electrode in contact with the tissue radially outward from the prospecting location in a first direction for a first honing distance;
observing no neural response to the electrical stimulation, translating the electrode in a second direction back to the prospecting location;
translating the electrode radially outward from the prospecting location in a third direction for a second honing distance; and
observing a neural response to the electrical stimulation.

28. A method according to claim 27, wherein the step of translating in the first direction is performed while applying the electrical stimulation to the tissue.

29. A method according to claim 27, wherein the second direction is opposite the first direction.

30. A method according to claim 27, wherein the first honing distance is substantially the same as the second honing distance

31. A method according to claim 27, wherein the second direction is substantially the same as the third direction.

32. A method according to claim 27, wherein the step of translating in the second direction is performed while the electrode is in contact with the tissue.

33. A method according to claim 27, wherein the step of translating in the third direction is performed while the electrode is in contact with the tissue.

34. A method according to claim 27, further comprising the step of identifying an active stimulation location on the tissue.

35. A method according to claim 27, further comprising the step of repeating at least once each of the steps of:
identifying a prospecting location;
applying electrical stimulation to the tissue at the prospecting location with an electrode to determine whether a neural response is generated; and
identifying an active stimulation location on the tissue.

36. A method according to claim 1, wherein the tissue region comprises subcutaneous tissue.

37. A method according to claim 7, wherein the tissue region comprises subcutaneous tissue.

38. A method according to claim 18, wherein the animal tissue comprises subcutaneous tissue.

39. A method according to claim 29, wherein the animal tissue comprises subcutaneous tissue.
